(19) United States
(12) Patent Application Publication
(10) Pub. No.: US 2025/0191685 A1
(43) Pub. Date: Jun. 12, 2025

(54) ZINC FINGER DESIGN USING A HIERARCHICAL MACHINE LEARNING MODEL

(71) Applicant: THE GOVERNING COUNCIL OF THE UNIVERSITY OF TORONTO, Toronto (CA)

(72) Inventors: Philip Mjong-Hyon Shin KIM, Toronto (CA); Osama ABDIN, Scarborough (CA)

(21) Appl. No.: 18/715,995

(22) PCT Filed: Dec. 2, 2022
(86) PCT No.: PCT/CA2022/051774

§ 371 Date: Jun. 3, 2024

Related U.S. Application Data
(60) Provisional application No. 63/285,282, filed on Dec. 2, 2021.

(51) Int. Cl. G16B 20/30; G16B 35/00; G16B 40/20

(52) U.S. Cl. CPC G16B 20/30; G16B 35/00; G16B 40/20

(57) ABSTRACT

A machine learning model for designing Zinc Finger proteins that bind to a given nucleic acid target sequence is described. The model uses a hierarchical architecture comprising a first layer trained on single-helix specificity data in a diverse set of interface environments and a second layer trained on dual helix specificity data.

### CROSS-REFERENCE TO RELATED APPLICATIONS

This application claims the benefit of U.S. Provisional Patent Application No. 63/285,282 for ZINC FINGER DESIGN USING A HIERARCHICAL MACHINE LEARNING MODEL, filed on 2 Dec. 2021, which is hereby incorporated by reference in its entirety.

### FIELD

The present application relates to the design of zinc fingers and more specifically to the use of a hierarchical machine learning model for the design of zinc fingers arrays.

### BACKGROUND

Programmable regulation of gene expression would offer both powerful research tools as well as enormous therapeutic potential. For instance, diseases caused by haploinsufficiency, gain of function mutations, or the misexpression of a gene can be directly treated by modifying gene expression(1-3). While CRISPR and TALE-based tools have been developed for such applications(4-9), their intrinsic characteristics could limit their therapeutic efficacy. For instance, the size of these proteins(10) complicates delivery, in particular using AAVs, the clinically best validated delivery method. Moreover, pre-existing immunity in humans for spCas9(11, 12) makes their long-term expression an immunogenic risk. Research applications designed to probe regulatory mechanisms could also be hampered by the difference in size and spatial arrangement of SpCas9 fusions compared to natural transcription factors (TFs): SpCas9 is 5-10 times the size of the most common DNA-binding domains (DBDs) found in human TFs, with the common C-terminal effector domain more than 6 times farther from the DNA(13, 14). In addition, the presentation of effector domains out of their natural context could impact their function. For example, it is unclear how the repressive potential of KRAB domains differs in isolation compared to when they are expressed in their parent proteins. Further, most synthetic activators use the viral VP16 domain(15, 16) or one of its derivatives which may not accurately mimic natural activating TFs that can encourage expression through different interactions and spatial arrangements. Finally, the effect of artificial regulators was shown to be highly dependent on binding position; differences as little as a single base can have a large impact(17), potentially restricting CRISPR-based tools due to their PAM limitations.

By contrast, the Cys2His2 zinc finger (ZF) domain offers unique advantages for targeting effector domains to desired genomic loci(18, 19). ZFs require less than 170 amino acids to specify a unique sequence in the human genome, enabling routine, even multiplexed delivery by AAVs. In addition, ZF domains are less likely than SpCas9 to be immunogenic as nearly 50% of human TFs use this DBD to specify their genomic targets(20). In fact, over 300 human ZF-TFs utilize KRAB domains(21) for repression while dozens of others are known to activate transcription(20, 22, 23).

While the potential utility of designer ZF arrays has long been recognized, engineering them has remained challenging with no proper design code having emerged thus far. This is not for lack of effort as multiple approaches have been used to generate ZF libraries(24-26) and ZF modules(27,28) to provide designer ZF arrays. However, these approaches either require multiple rounds of laborious selection that produce ZFs with inconsistent activity or the application of pre-selected modules that often fail when expressed out of their selected context. Conversely, a proper code for ZF array design could enable the reprogramming of natural ZF transcription factors to provide tools that can activate or repress target genes, that are small enough for multiplexed delivery in AAVs, with minimal risk of immunity.

Various approaches have been used to try and engineer ZFs with novel specificity. One approach focuses on engineering single helices or pairs of helices by selecting functional variants from ZF libraries. However, for a pair of ZF helices, there are about 4.1×10¹⁵ possible sequences and about 4.8×10⁸ orders in which each sequence can be generated. Enumerating all possibilities to is thus computationally intractable.

This combinatorial explosion problem thus reflects a technological problem, and improved systems and methods are desired in order to predict ZFs amino acid residues which can bind to a target nucleic acid.

### SUMMARY

A complex zinc finger screen was used to develop an Al-based model to design zinc finger arrays that bind to a target nucleic acid sequence. As set out in the Examples, a hierarchical machine learning model architecture was developed in order to capture interactions between neighboring ZF domains. Notably, the ZF protein design model was demonstrated to allow for the design of ZF arrays based on a target nucleic acid sequence with greater binding specificity than alternative models. Various transcription factors were then reprogrammed using the model by replacing DNA binding domains in order to target novel genomic sequences. The ZF protein design model can therefore be used to design molecules with novel binding specificities in order to regulate gene expression.

Accordingly, in one aspect there is provided a method for determining a Zinc Finger (ZF) protein sequence for binding a target nucleic acid sequence. In one embodiment, the method comprises providing a ZF protein design model and predicting a ZF protein sequence based on the ZF protein design model and the target nucleic acid sequence. In one embodiment, the ZF protein design model is a machine learning model trained on single helix binding data and helix-pair binding data. In one embodiment, the single helix binding data comprises binding data on single ZF helices that bind a nucleotide 4-mer and the helix pair binding data comprises binding data on ZF helix pairs that bind a nucleotide 7-mer.

In one embodiment, the method for determining a ZF protein sequence for binding a target nucleic acid sequence comprises:
providing, in a memory, a ZF protein design model comprising a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence;
receiving as input a target nucleic acid sequence at a processor in communication with the memory, the target nucleic acid sequence comprising the first target nucleic acid subsequence and the second target nucleic acid subsequence, wherein the first target nucleic acid subsequence and the second target nucleic acid subsequence overlap;
determining a first embedding at the processor, the first embedding based on the first target nucleic acid subsequence and the first module of the protein design model;
determining a second embedding at the processor, the second embedding based on the second target nucleic acid subsequence and the second module of the protein design model; and
determining the ZF protein sequence at the processor, the ZF protein sequence based on the first embedding, the second embedding, and the third module of the protein design model.

In another aspect, there is provided a system for determining a Zinc Finger (ZF) protein sequence for binding a target nucleic acid sequence. In one embodiment, the system comprises memory and a processor in communication with the memory for performing a method as described herein for determining a ZF protein sequence. In one embodiment, the system comprises:
a memory, the memory comprising:
   a ZF protein design model comprising a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence;
a processor in communication with the memory, the processor configured to:
   receive as input a target nucleic acid sequence, the target nucleic acid sequence comprising the first target nucleic acid subsequence and the second target nucleic acid subsequence, wherein the first target nucleic acid subsequence and the second target nucleic acid subsequence overlap;
   determine a first embedding based on the first target nucleic acid subsequence and the first module of the protein design model;
   determine a second embedding based on the second target nucleic acid subsequence and the second module of the protein design model; and
   determine the ZF protein sequence based on the first embedding, the second embedding, and the third module of the protein design model.

In another aspect, there is provided a method of generating a model for determining a Zinc Finger (ZF) protein sequence for binding a target nucleic acid sequence. In one embodiment, the method comprises:
providing a hierarchical machine learning model comprising:
   a first layer comprising a first module and a second module; and
   a second layer comprising a third module,
   wherein embeddings from the first layer are fed into the second layer;
training the first module based on single helix binding data, wherein the single helix binding data comprises data on single ZF helices that bind polynucleotides comprising a target 3-mer and one or more adjacent nucleotides;
training the second module based on single helix binding data, wherein the single helix binding data comprises data on single ZF helices that bind polynucleotides comprising a target 3-mer and one or more adjacent nucleotides; and
training the hierarchical machine learning model based on helix-pair binding data, wherein the helix pair binding data comprises data on ZF-helix pairs that bind polynucleotides comprising a target 6-mer.

### DRAWINGS

Various embodiments will now be described in relation to the drawings in which:

FIGS. 1A-E shows an overview of interface-focused ZF screens. (A) Structure of adjacent ZF domains showing their close proximity. Helical position 6 of domain 1 and position −1 of domain 2 are outlined. (B) Cartoon of interactions between adjacent helices and the DNA. The six helical positions of the three domains are shown as circles with the common contacts made by positions −1, 2, 3. and 6 indicated with arrows. The overlap environment, that includes the base adjacent to the library interaction and the amino acid used to specify that base, is highlighted in green. This environment is unique for each library. (C) Cartoon of the B1H selections. The 3-fingered protein is expressed as a C-terminal fusion to the omega subunit of RNA polymerase. For each library, ZF domain 2 is randomized at six helical positions and screened for amino acid combinations able to specify each of the 64 possible “NNN” targets. This is done in 64 independent screens. Domains 0 and 1 bind to their known, preferred targets, and thereby present an overlap environment that is unique to the library. Only helices able to bind the target in the unique library overlap environment will recruit the polymerase. activate the reporter, and survive on selective media. (D) (left) The helical residues for domains 0, 1, and 2 are shown for each library screened. Domain 2 contains all possible combinations of the six helical residues. Domain 1 is fixed in the selections but varied by library. The 6th residue of domain 1 is the side chain that will be exposed at the interface between domains 1 and 2. Domain 0 is the same in all libraries except library 1. (right) There are 64 DNA targets for domain 2 to be screened against in 64 independent selections. The fixed targets for domain 1 of each library are shown with the overlap base in bold. (E) (left) To assay the success of each selection we determined clusters from the data and used the maximum information content at one position of a cluster to provide a relative measure of enrichment across all selections. (right) Molecular dynamic simulations were performed on all domain 1 helices in their previously characterized contexts. The number of suggested contacts between domain 1 and the DNA are shown for each library.

FIGS. 2A-E show that specificity solutions are library-specific. (A) (top) A dot plot comparison of 1-Hamming distance is provided comparing the similarity of helical strategies enriched in libraries 1 thru 9 for three G-rich targets (right) and three G-poor targets (left). The darkness of the dot represents the similarity of the enriched populations with dark dots being more similar. Empty spots indicate a failed target selection for one or both of the libraries compared. (bottom) Normalized hamming distance for all libraries across all targets listed from least similar (left) to most similar (right). The targets compared above include G-poor targets 202 (CTT, TTC and ATC) and G-rich targets 204 (CGG, CTG, GGG). (B) Clusters were determined by MUSI from the enriched helices in each library selection. Three clusters are shown for 4 different binding sites (CCA, TTT, CCG, and GAG). If a cluster was enriched in a library selection the corresponding box is filled black in the table. (C) Schematic illustration (top) and molecular dynamics snapshot (bottom) of the hydrogen bonds between the arginine at position 2 of the domain 2 helix QsRYtt with the G* of the CCG* target when an asparagine is at position 6 of the adjacent finger (Library 2 environment) or when an arginine is at position 6 of the adjacent finger (Library 3 context). (D) (top) Cartoon of B1H2-finger selections. (bottom) The number of helices enriched in the 2-finger selections is shown as a factor of the number of single finger libraries they originated in. (E) A comparison of the helices enriched in the 2-finger selections shows the average number of single finger libraries from which a helix originated in by binding site.

FIGS. 3 shows an interface-focused zinc finger design model. The model is composed of two modules that are helix B1H selections to predict residues in partially masked helices. The partially masked helices bind 4-mer nucleotide generated residues embeddings from these modules are fed into a third module that learns compatibility. The full model is trained on two-helix B1H selection data to predict residues that partially bind 7-mer nucleotides sequences.

FIGS. 4A-G shows the performance of two-helix design model. (A) Training and validation accuracy during pre-training step. (B) Training and validation accuracy during fine-tuning step. (C) Helix sequence reconstruction accuracy with different numbers of masked residues. (D) Comparison of differences between predicted and real selection logos using the developed model and ZFPred. (E) Comparison of differences between predicted and real selection logos using the two-helix model and concatenated logos from the single-helix design model. (F) Comparison of differences between predicted and real selection logos using the two-helix model and concatenated logos from the single-helix B1H selections. (G) Predicted logos, real B1H logos, and concatenated single-helix B1H logos for test set sequences.

FIGS. 5A-E show zinc finger designed nucleases. (A) ZFNs bind DNA as dimers in a tail-to-tail orientation, spaced by 5 or 6bp. The cartoon shows each monomer with two pairs of ZFs separated by a base-skipping linker, for a total 8-finger ZFN. (B) A comparison of loss of fluorescence in a GFP disruption assay for 8-finger ZFNs that were either selected or designed to cut the same targets. (C) Substitution of 2 of the 8-fingers in designed arrays with selected fingers increase activity. (D) Sixteen 12-finger ZFNs, 6 per monomer, are tested for loss of fluorescence. (E) A six-finger array was designed to bind a repeat sequence on chromosome 14, expressed as a GFP fusion, and visualized by live cell imaging.

FIGS. 6A-G show reprogrammed transcription factors. (A) The ZFs of KLF6 are seamlessly replaced with designed ZFs. (B) A GFP reporter is activated with 4 ZF designs to bind the TetO sequence. (C) Comparison of RTFs to the rTetR-VP64 activator using the Tet3 ZF array. (D) The ZFs of 4 KRAB TFs are replaced with the Tet3 ZF array and challenged to repress a constitutive GFP reporter. (E) Repression of endogenous targets with Zim3 RTFs measured by RT-qPCR. (F) Left, relative expression of CDKN1C by KLF6 RTFs with 7 ZF arrays designed to bind sequences upstream of the TSS. Right, a comparison of the CDK #200 array with phosphate modifications at CDKN1C and two off-target sequences. (G) Structure, substitution of a phosphate contacting residue (shown in box 602) can reduce nonspecific affinity. Right, table of misregulated genes by phosphate modification. Below, a comparison of RNA-seq data for 0 and 8 phosphate-contacting modifications.

FIGS. 7A-C show zinc finger interface and common selection strategies. (A) Cartoon of two adjacent fingers interacting with DNA. The six positions of the helix with base-specifying potential are shown. Position 4 is not shown as it is typically a hydrophobic residue that packs into the core of the domain. It is not randomized in any selection schemes. The interface and overlap contacts are highlighted with an oval. (B) Cartoon of a single finger selection approach where all the randomization is on one of the two fingers(24, 26, 27, 29-32, 52). These were mostly done with an arginine-guanine contact (highlighted) adjacent to the selected finger(24, 26, 27, 29-32) or, in one case, where the library was the N-terminal finger(27). On the randomized helix the letter's in bold and red (CFWY) were not coded for in the OPEN and other zinc finger libraries(26, 27, 30, 31). (C) Two versions of libraries that selected interface interactions are shown. Top. Many of the contacts were fixed with 5 positions incompletely randomized. The red and bold amino acids were not available in these libraries(28, 33). Bottom. Another approach randomized more positions but used a very small subset of amino acids. Only available amino acids are listed(25, 53, 54).

FIG. 8 shows cartoons that depict what environment is presented to the selected zinc finger in each library with A overlaps on the left, C overlaps on the right, and G overlaps at the bottom.

FIGS. 9A-C shows the performance of single-helix design modules. (A) Training and validation accuracy during pre-training step. (B) Helix sequence reconstruction accuracy with different numbers of masked residues. C) Comparison of differences between predicted and real selection logos using the developed model and ZFPred.

FIGS. 10A-E shows the distribution of target sequences in the training and validation datasets. (A) Graph representation of the seven-mer sequences in the training and validation datasets. Nodes represent seven-mers and edges connect nodes representing sequences within two substitutions of each other. Orange nodes are validation set sequences; blue nodes are training set sequences. (B) Distances of validation set sequences to training set sequences. (C) Distances of test set sequences to training set sequences. (D) Distances of all seven-mer sequences to training set sequences. (E) Distances of all seven-mer sequences to all sequences against which selections were performed.

FIG. 11 shows quantification of the effect of pre-training on model performance. (A) Comparison of reconstruction accuracies when the model is pre-trained on single-helix selections and re-trained, re-trained with parameters of the single-helix modules frozen, and not pre-trained. (B) Comparison of the perplexities when the model is pre-trained on single-helix selections and re-trained, re-trained with parameters of the single-helix modules frozen, and not pre-trained.

FIG. 12 shows the impact of the number of generated samples on maximum likelihood design using A* or temperature dependent sampling.

FIG. 13 shows likelihood penalty when moving from skip-base to extended array designs. (A) A schematic showing the difference between skip-base and extended array constructs. (B)-(F) Likelihood values for helix pairs one to five in the top scoring six-helix extended array constructs compared to the top scores for individual helix pairs generated for their respective target seven-mers.

FIG. 14 shows ZF designed nucleases. (A) Loss of GFP fluorescence using designed extended ZFs. (B) T7 endonuclease assay using designed extended ZFs against CCR5.

FIG. 15 shows ZF designed repressors. (A) Western blot against SNCA using cells treated with extended ZF-ZIM3 repressors designed against different regions around the TSS of SNCA. (B) RT-qPCR using cells treated with extended ZF-ZIM3 repressors designed against different regions around the TSS of mouse SNCA. (C) qPCR using cells treated with extended ZF-ZIM3 repressors designed against different regions around the TSS of TDP43.

FIG. 16 shows a system diagram for determining a ZF protein sequence for binding a target nucleic acid sequence, and optionally for generating a model for determining a ZF protein sequence. in accordance with one or more embodiments.

FIG. 17 shows a device diagram of a server for determining a ZF protein sequence for binding a target nucleic acid sequence, and optionally for generating a model for determining a ZF protein sequence, in accordance with one or more embodiments.

FIG. 18A shows a ZF design model diagram in accordance with one or more embodiments.

FIG. 18B shows a method diagram for determining a ZF protein sequence for binding a target nucleic acid sequence, in accordance with one or more embodiments.

FIG. 19 shows a method diagram for reprogramming a biomolecule to bind a target nucleic acid sequence, in accordance with one or more embodiments.

FIG. 20 shows a method diagram for generating a model for determining a Zinc Finger (ZF) protein sequence for binding a target nucleic acid sequence, in accordance with one or more embodiments.

FIG. 21 shows an exemplary user interface for ZFDesign, in accordance with one or more embodiments.

FIG. 22 shows exemplary sequences in accordance with one or more embodiments.

FIG. 23 shows exemplary sequences in accordance with one or more embodiments.

### DESCRIPTION OF VARIOUS EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the diagrams are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer. a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to at least one output device, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks. tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modifications and variations may be made to these example embodiments without departing from the spirit and scope of the invention, which is limited only by the appended claims. Also, in the various user interfaces illustrated in the figures. it will be understood that the illustrated user interface text and controls are provided as examples only and are not meant to be limiting. Other suitable user interface elements may be possible.

In one aspect, there is provided a system and method for determining a ZF protein sequence for binding a target nucleic acid sequence. The embodiments described herein allow for the design of ZF nucleic acid binding domains based on a target nucleic acid sequence. As shown in the Examples, the use of a hierarchical machine learning model explicitly designed to capture the interactions between neighboring zinc finger domains has been demonstrated to effectively produce highly functional ZF arrays with strong on-target activity.

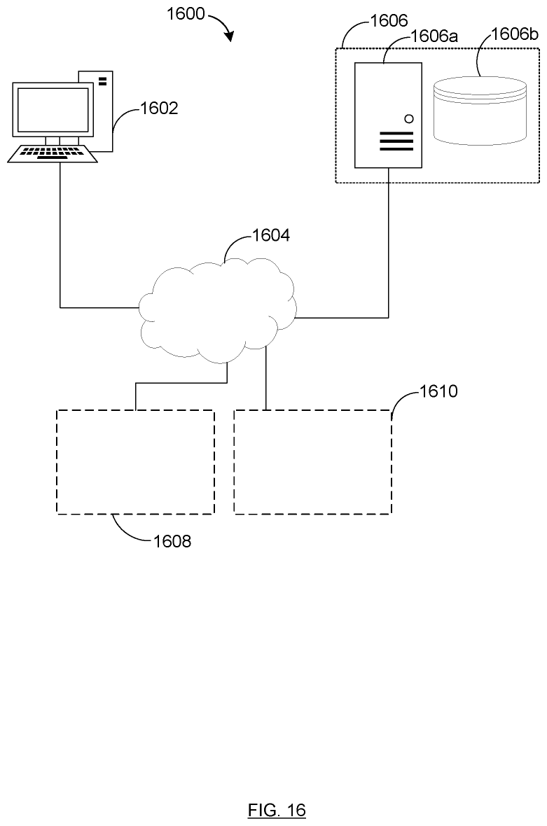

Referring to FIG. 16, there is shown a system diagram of a ZF prediction system 1600 for determining a ZF protein sequence for binding a target nucleic acid sequence, and optionally for generating a model for determining a ZF protein sequence, in accordance with one or more embodiments. The ZF prediction system 1600 includes one or more user devices 1602, a network 1604 and a computing device 1606.

The one or more user devices 1602 may be used by a user such as an administrator, geneticist or technician to access a software application (not shown) running on server 1606a at remote service 1606 over network 1604. In one embodiment, the one or more user devices 1602 may access a web application hosted at server 1606a using a browser for determining a ZF protein sequence for binding a target nucleic acid sequence. In an alternate embodiment, the one or more user devices 1602 may download an application (including downloading from an App Store such as the Apple® App Store or the Google® Play Store) for determining a ZF protein sequence for binding a target nucleic acid sequence.

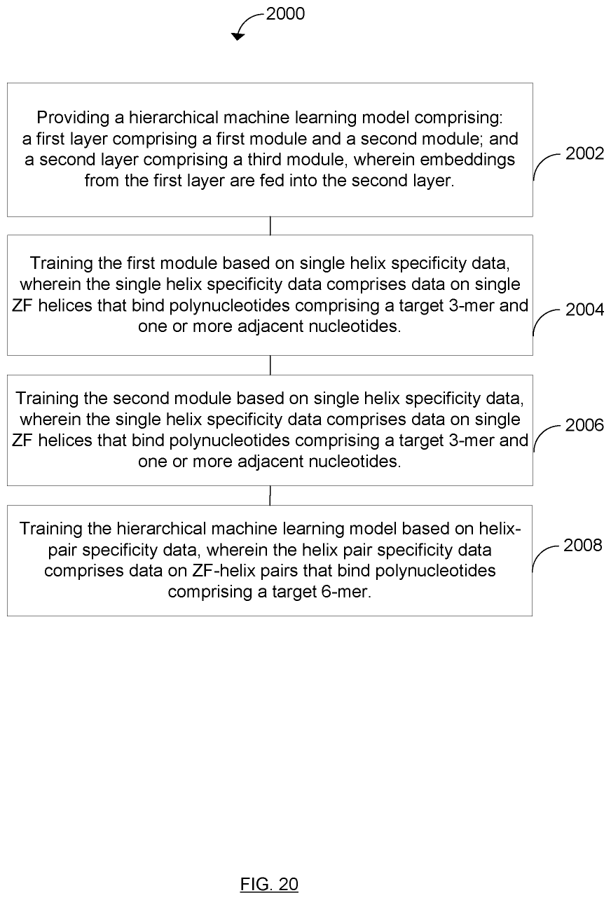

In an alternate embodiment, the one or more user devices 1602 may operate the remote service 1606 (either using the web application or via a downloaded application) to generate a ZF design model. This may include generating a ZF design model based on the method of FIG. 20.

The one or more user devices 1602 may be any two-way communication device with capabilities to communicate with other devices. A user device 1602 may be a desktop computer, mobile device, or laptop computer. A user device 1602 may be a mobile device such as mobile devices running the Google® Android® operating system or Apple® iOS® operating system. A user device 1602 may be the personal device of a user, or may be a device provided by an employer.

The one or more user devices 1602 may be used by an end user to access the software application running on server 1606a over network 1604. In one embodiment, the one or more user devices 1602 may access a web application hosted at server 1606a and may allow a user to review a ZF protein sequence prediction in a database at data store 1606b. including historical ZF protein sequence predictions.

The user at the one or more user devices 1602 may send or submit a nucleic acid sequence to be targeted to the ZF prediction system running on the remote service 1606. The target nucleic acid sequence may be sent in a ZF prediction request. The ZF prediction request may be a web application request, an Application Programming Interface (API) request, or another request. The nucleic acid target sequence may be provided in a variety of formats in the ZF prediction request. For example, the nucleic acid target sequence may be provided by the user, optionally in plain sequence format, FASTQ format, EMBL format, or FASTA format. or in other similar formats containing nucleic acid sequence information. Alternatively, the nucleic acid target sequence may be manually entered through user device 1602 or by entering or referencing an accession number or database entry corresponding to the sequence for a nucleic acid target sequence. Upon receipt of the ZF prediction request, the ZF prediction system on remote service 1606 may use the methods described herein in order to determine a ZF prediction, and transmit the ZF prediction to the user at user device 1602. The ZF prediction response may be provided to the one or more user devices 1602 in an email, as a notification, or text message. The ZF predictions may include a sequence of amino acids corresponding to one or more ZF residue positions, consecutive or non-consecutive, that are responsible for nucleic acid binding activity. The ZF prediction may be limited to residues that define or form all or part of the DNA binding domain, or the ZF prediction may include all of part of a ZF backbone sequence. In one embodiment, the ZF prediction is a probability distribution over amino acids for the 6 DNA-binding residues for each helix.

The software application running on the one or more user devices 1602 may display one or more user interfaces on a display device of the user device.

Network 1604 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The remote service 1606 may include a server 1606a and a database 1606b. The server 1606a may further be in communication with a database 1606b. The database 1606b and the server 1606a may be provided on the same server device, may be configured as virtual machines, or may be configured as containers. The database 1606b may be provided by server 1606a, another server (not shown), or a cloud-based database service such as Amazon Web Services® (AWS). The remote service 1606 may itself be provided by a service such as AWS. The remote service 1606 is in network communication with the one or more user devices 1602.

The server 1606a may host a web application or an Application Programming Interface (API) endpoint that the one or more user devices 1602 may interact with via network 1604. The server 1606a may make calls to the database 1606b to query ZF interaction data, ZF protein design model data, ZF library data, or other data. The requests made to the API endpoint of server 1606a may be made in a variety of different formats, such as JavaScript Object Notation (JSON) or extensible Markup Language (XML).

The database 1606b may store information including ZF interaction data from a plurality of ZF interaction libraries (including both one-finger and two-finger interaction data as described herein), user data, ZF design model data including pre-trained models. The database 1606b may be a Structured Query Language (SQL) such as PostgreSQL or MySQL or a not only SQL (NoSQL) database such as MongoDB.

In one embodiment, the server 1606a may perform model training of the ZF design model, as described in FIG. 20. The model training by server 1606a may include querying the database 1606b for ZF interaction data, including both one-finger and two-finger interaction data for use in model training as described herein.

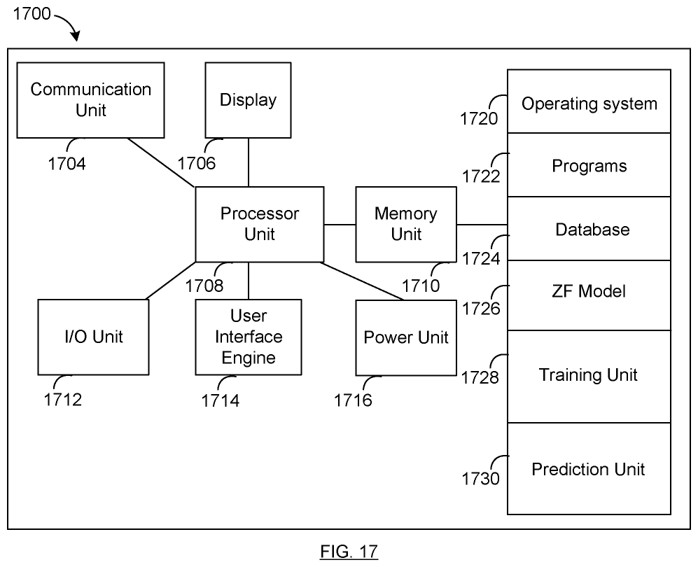

Referring next to FIG. 17 there is a device diagram 1700 of a server (for example server 1606a in FIG. 16) for determining a ZF protein sequence for binding a target nucleic acid sequence. and optionally for generating a model for determining a ZF protein sequence, in accordance with one or more embodiments. The server 1700 includes a communication unit 1704, a display unit 1706, a processor unit 1708, a memory unit 1710, an I/O unit 1712, a user interface engine 1714, and a power unit 1716.

The communication unit 1704 can include wired or wireless connection capabilities. The communication unit 1704 can include a radio that communicates using standards such as IEEE 802.11a, 802.11b, 802.11g, or 802.11n. The communication unit 1704 can be used by the server 1700 to communicate with other devices or computers.

Communication unit 1704 may communicate with a network, such as network 1604 (see FIG. 16).

The display 1706 may be an LED or LCD based display, and may be a touch sensitive user input device that supports gestures.

The processor unit 1708 controls the operation of the server 1700. The processor unit 1708 can be any suitable processor, controller or digital signal processor that can provide sufficient processing power depending on the configuration, purposes and requirements of the server 1700 as is known by those skilled in the art. For example, the processor unit 1708 may be a high performance general processor. In alternative embodiments. the processor unit 1708 can include more than one processor with each processor being configured to perform different dedicated tasks. The processor unit 1708 may include a standard processor. such as an Intel® processor or an AMD® processor. In one embodiment, processor unit 1708 may comprise one or more Graphic Processing Units (GPUs), such as but not limited to a Compute Unified Device Architecture (CUDA)-compatible GPU with 4 GB of VRAM or higher.

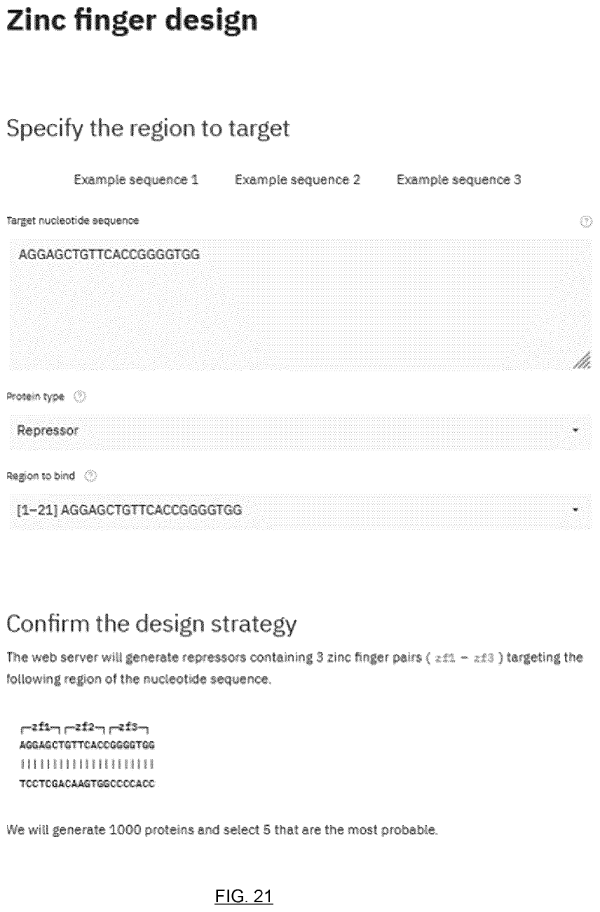

The processor unit 1708 can also execute a user interface (UI) engine 1714 that is used to generate various UIs for delivery via a web application, an example of which is shown in FIG. 21.

The memory unit 1710 comprises software code for implementing an operating system 1720, programs 1722, database 1724, ZF model 1726, training module 1728, and prediction module 1730.

The memory unit 1710 can include RAM, ROM, one or more hard drives, one or more flash drives or some other suitable data storage elements such as disk drives, etc. The memory unit 1710 is used to store an operating system 1720 and programs 1722 as is commonly known by those skilled in the art.

The I/O unit 1712 can include at least one of a mouse, a keyboard, a touch screen, a thumbwheel, a track-pad, a track-ball, a card-reader, an audio source, a microphone, voice recognition software and the like again depending on the particular implementation of the server 1700. In some cases, some of these components can be integrated with one another.

The user interface engine 1714 is configured to generate interfaces for users to request model training, request ZF predictions based on a target nucleic acid sequence, review ZF interaction data, or other activities associated with training and ZF prediction. The various interfaces generated by the user interface engine 1714 may be transmitted to a user device via the communication unit 1704.

The power unit 1716 can be any suitable power source that provides power to the server 1700 such as a power adaptor or a rechargeable battery pack depending on the implementation of the server 1700 as is known by those skilled in the art.

The operating system 1720 may provide various basic operational processes for the server 1700. For example, the operating system 1720 may be a server operating system such as Ubuntu® Linux, Microsoft® Windows Server® operating system, or another operating system.

The programs 1722 include various user programs. They may include several hosted applications delivering services to users over the network, for example. a web application and an API application, and other applications as known.

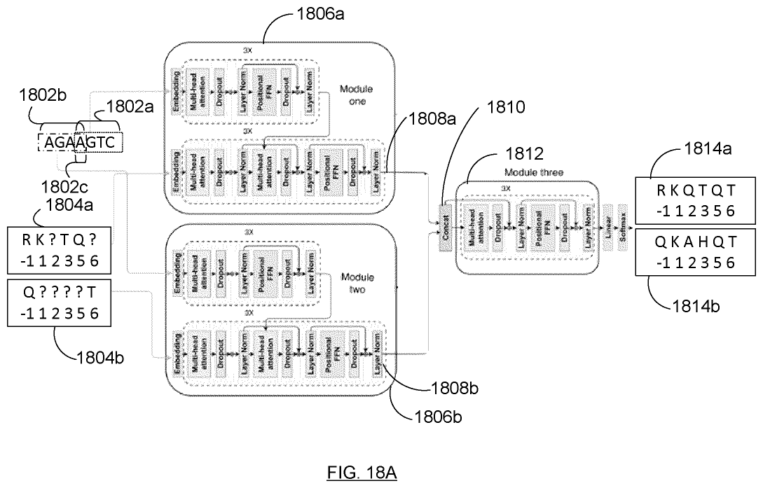

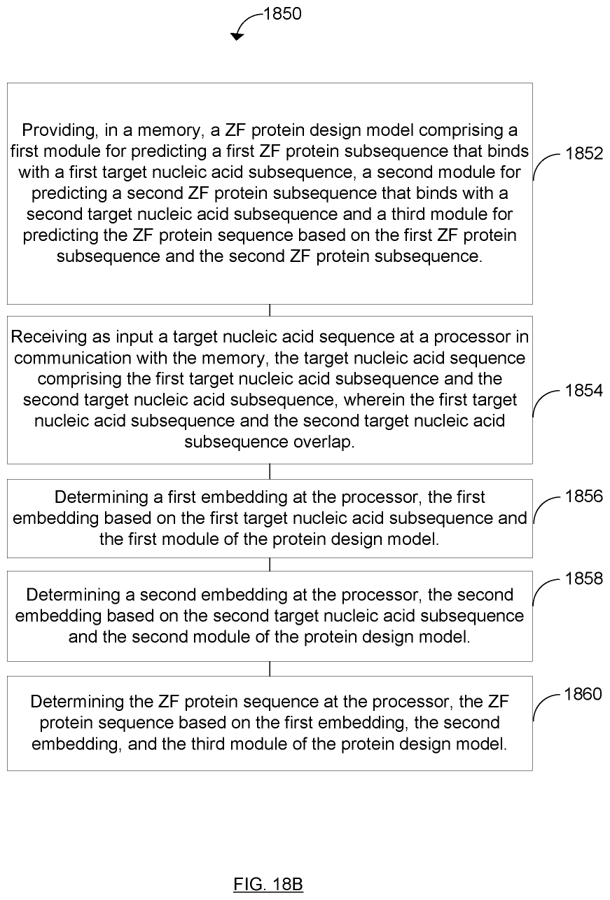

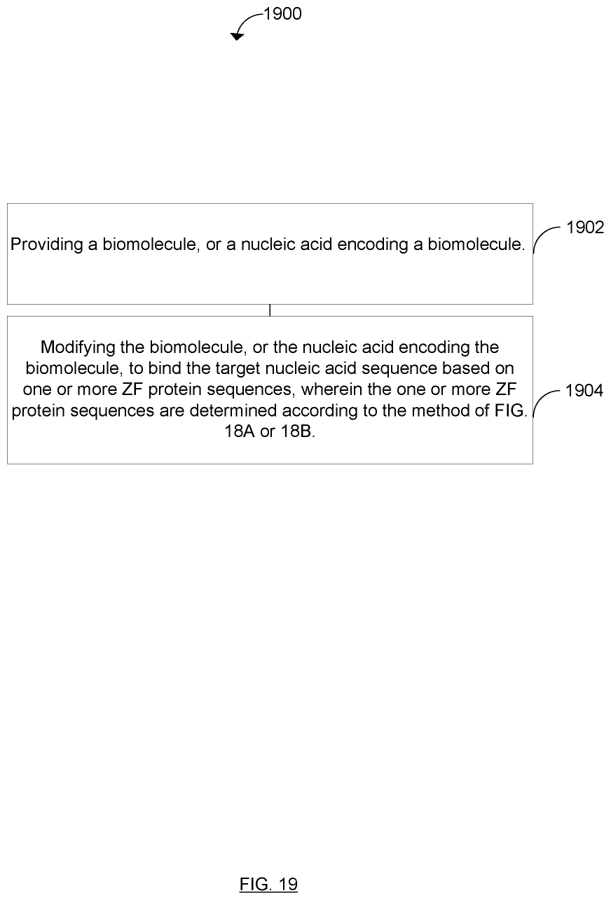

In one or more embodiments, the programs 1722 may provide an application such as a web-based application for ZF prediction, or client-server based application via an API. The application may provide functionality for a user to submit a ZF prediction request including a target nucleic sequence that may initiate the ZF prediction methods described in FIG. 18. The application may provide functionality for a user to submit a biomolecule synthesis request in order to synthesize a biomolecule, such as polypeptide, comprising one or more nucleic acid binding ZF arrays determined based on the methods described in FIG. 19. The application may provide functionality for a user to submit a model generation request for a ZF design model that may initiate the ZF model training methods described in FIG. 20. The application may further provide access for queries and data analysis of the ZF interaction data in database 1724, ZF design models in database 1724, ZF library data, or other data.

The database 1724 may be a database for storing ZF design model data and ZF interaction data, such as but not limited to binding data from both one-finger (helix) and two-finger (helix) libraries.

Database 1724 may include binding data from a plurality of different libraries comprising binding data associating ZF motifs and corresponding nucleic acid target sequences. In one embodiment, database 1724 contains binding data for single helix ZF motifs for all 64 possible 3-mers under a range of influences at the interface where adjacent finger specificity may overlap. In one embodiment, database 1724 contains binding data for two-finger (dual-helix) motifs that is representative of selected single helix ZF motifs that are compatible (i.e. have binding activity) in different adjacent finger contexts.

For example, as set out in the Examples one-finger libraries may include interaction data between ZFs and 3 or 4 base-pair nucleic acid sites. The two-finger interaction libraries may include interaction data between ZFs and 6 or 7 base-pair nucleic acid sites.

The ZF design model (including the first module, the second module, and the third module) may be trained and/or evaluated on empirical data, such as data derived from methods known in the art for identifying the sequence-specific target site of a DNA-binding domain, such as but not limited to bacterial one-hybrid (B1H) systems. Screening data such as B1H data may be processed or filtered, such as where helices are evaluated based on the diversity of encoding nucleotide sequences found in the screen. The Shannon entropy for each helix (or helix pair) may be determined based on the number of reads associated with each possible encoding nucleotide sequence. Helices may be filtered based on predetermined thresholds. For example, in one embodiment helices with less than ten reads or a Shannon entropy of less than 0.07 may be removed.

Modules one and two may be pre-trained using data from single-helix B1H selections that were performed against nucleotide four-mers. For example. the data used in Example 1 included selections performed with 11 libraries against 192 different nucleotide four-mers such that the dataset included over 2 million data points. For initial training and hyperparameter tuning, the data points may be split into train, test, and validation datasets. For example, the data used in Example 1 was split into train, test and validation data sets at proportions of 80%, 10%, and 10% respectively by four-mers sequence. For pre-training, the data may be instead split by helix sequence. In one embodiment, modules one and two are trained using data on nucleotide 4-mers that exhaustively samples all 64 possible nucleotide 3-mers with a variety of adjacent 3′ nucleotides.

In one embodiment, the ZF protein design model, including a pre-trained first module, a pre-trained second module and a third module for predicting the ZF protein sequence based on the first ZF protein subsequence and the second ZF protein subsequence, is further trained using helix-pair binding data. In one embodiment the ZF protein design model may be trained using data from helix-pair B1H selections that were performed against nucleotide seven-mers.

While the number of possible nucleotide sever-mers is significant (16,384), the machine learning model described herein may be effectively trained using a subset of dual-helix target sequences as demonstrated in the Examples. In Example 1 an initial dataset of selections against 189 seven-mers containing a total of 327,792 data points was split into training and validation datasets at proportions of 90% and 10%. In one embodiment, the machine learning model described herein is trained using helix-pair binding data generated using at least 100, 150, 200, 250, 300, 350 or 400 sever-mers. Alternatively or in addition, the machine learning model described herein is trained using a total of 150,000, 200,000, 250,000, 300,000 400,000 or 500,000 data points.

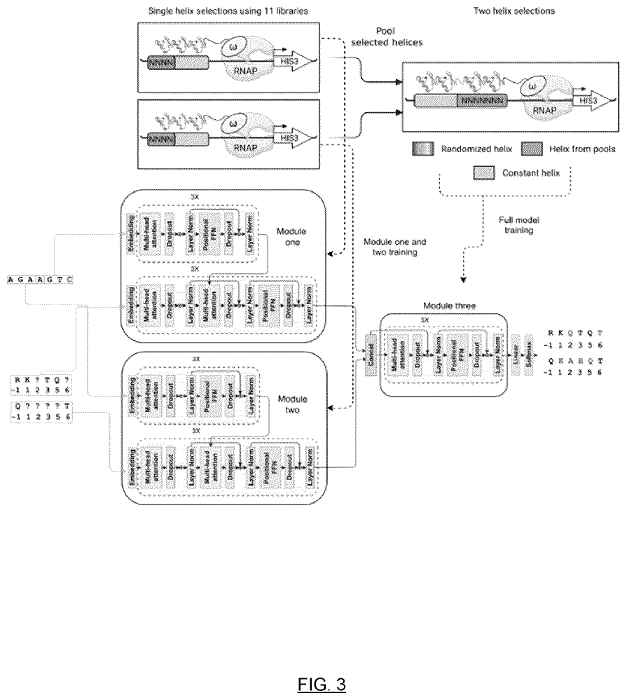

In one embodiment, ZF model 1726 is a machine learning model such as a neural network. The ZF model 1726 may be the hierarchical model in FIG. 3, including a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence. The ZF model may be operated by the prediction module 1730 based on a received ZF prediction request.

The training module 1728 is for generating the machine learning model (such as a neural network) for operation by the ZF model 1726, and for storage in database 1724. The training module 1728 may perform methods as described herein in FIG. 20. Once the model is generated by the training module 1728, it may be stored in the database 1724 or used by the ZF model 1726.

There may be generally two training steps, and in both, a nucleotide target and a sequence of partially masked core residues from either a single zinc finger helix or a helix pair may be provided to the model. For example, a fraction of the core resides (e.g. 50%) may be masked and the cross-entropy loss may be evaluated based on the output probabilities. Training may be done using an Adam optimizer as set out in the Examples. Early stopping may be done based on the validation loss. Pre-training modules one and two may take many iterations, followed by additional iterations for training the full model (including both layers). When training the full model, the parameters for modules one and two may be either randomly initialized, transferred from the pre-training step, or transferred from the pre-training step and frozen.

The prediction module 1730 may determine ZF predictions using the ZF model 1726. The ZF predictions may be generated by the prediction module 1730 and stored in database 1724, or transmitted to a user at a user computing device. A user may then use the ZF prediction to design or synthesize a nucleic acid binding domain in a protein molecule, optionally by reprogramming existing transcription factors or other biomolecules to target a particular nucleic acid sequence.

Referring next to FIGS. 18A and 18B together there are shown a machine learning model diagram 1800 and method diagram 1850 respectively for determining a ZF protein sequence for binding a target nucleic acid sequence, in accordance with one or more embodiments.

At 1852, a ZF protein design model is provided in a memory, the ZF protein design model comprises a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence. The ZF protein design model may be, for example, the ZF protein design model 1800, including a first module 1806a, a second module 1806b. and a third module 1812. The ZF protein design model may be loaded into memory from a database. The ZF protein design model may be trained based on the method described in FIG. 20. The ZF protein design model may be a hierarchical model, including the first module 1806a, the second module 1806b, and the third module 1812. The first module 1806a and the second module 1806b may operate in a first layer, and the third module 1812 may operate in a second layer.

At 1854, a target nucleic acid sequence 1802 is received as input at a processor in communication with the memory, the target nucleic acid sequence comprising the first target nucleic acid subsequence 1802a and the second target nucleic acid subsequence 1802b. wherein the first target nucleic acid subsequence 1802a and the second target nucleic acid subsequence 1802b overlap 1802c. For example, as shown the first target nucleic acid subsequence 1802a may be ‘AGTC’, the second target nucleic acid subsequence 1802b may be ‘AGAA’ and the overlap portion 1802c may be ‘A”. In other words, the 5′ nucleotide of first target nucleic acid subsequence 1802a may overlap with the 3′ nucleotide of second target nucleic acid subsequence 1802b. The target nucleic acid sequence 1802 may be received from a user at a user device. In one embodiment, target nucleic acid sequence 1802 comprises or consists of a sequence of 7 nucleotides made up of a first target nucleic acid subsequence of 4 nucleotides and a second nucleic acid subsequence of 4 nucleotides.

At 1856, a first embedding 1808a is determined at the processor, the first embedding 1808a based on the first target nucleic acid subsequence 1802a and the first module 1806a of the protein design model 1800.

At 1858, a second embedding 1808b is determined at the processor, the second embedding 1808b based on the second target nucleic acid subsequence 1802b and the second module 1806b of the protein design model 1800.

At 1860, a ZF protein sequence 1814 is determined at the processor, the ZF protein sequence 1814 based on the first embedding 1808a, the second embedding 1808b, and the third module 1812 of the protein design model 1800. In one or more embodiments, the methods and system described herein are useful for determining ZF protein sequences for a pair of adjacent ZF helices, for example ZF protein sequences 1814a and 1814b that define residues that bind to first and second target nucleic acid subsequences 1802a and 1802b.

Without being limited by theory, it is believed that a hierarchical machine learning model as described herein is particularly effective at capturing the relationships between amino acid residues in ZF nucleic acid binding motifs, including adjacent-finger influences on binding activity.

In one or more embodiments, the first module 1806a, the second module 1806b. and/or the third module 1812 may comprise attention-based deep learning models.

In one or more embodiments, the first module 1806a, the second module 1806b. and/or the third module 1812 may comprise recurrent neural networks (RNN), optionally recurrent neural networks with long short-term memory.

In one or more embodiments, the first module 1806a, the second module 1806b. and/or the third module 1812 may comprise convolutional neural networks (CNN).

In one or more embodiments, the first module 1806a and the second module 1806b may comprise encoder models and the third module 1812 comprises a decoder model, optionally wherein the encoder models generate a high-dimensional representation for each DNA base in the target nucleic acid sequence and the decoder model generates predictions for each amino acid residue in the ZF protein sequence 1814.

In one or more embodiments, the first embedding 1808a and the second embedding 1808b may be concatenated prior to input to the third module 1812.

In one or more embodiments, the third module 1812 may comprise at least one self-attention layer and at least one feed forward layer.

In one or more embodiments, the at least one self-attention layer may comprise at least three self-attention layers, the at least one feed forward layer comprises at least three self-attention layers, and each self-attention layer comprises at least four heads.

In one or more embodiments, the concatenation 1810 of the first embedding 1808a and the second embedding 1808b may comprise an embedding dimension of 128; a value and a key embedding for computing scaled dot-product attention in the at least one self-attention layers comprises 256 dimensions; and/or a hidden dimension in the at least one feed-forward layers comprises 128 dimensions.

When predicting zinc finger residues, the model may make use of context provided by known residues. Helix sequences may be generated incrementally where the model is run once for each missing residue. For example, the ZF sequences 1804 may be incrementally predicted to identify the unknown residues (indicated with a ‘?’ in FIG. 18A). At each iteration. a single residue may be added to increase the sequence context.

To generate sequences, various search algorithms known in the art such as the A* algorithm or Monte Carlo sampling may be used. This approach may involve iteratively filling in masked residues 1814 while maintaining a priority queue of partially masked sequences 1804. At every iteration, the top partially masked sequence may be taken from the priority queue and passed through the model. All possible labels for every masked residue may be evaluated. Any label with a probability above a set threshold (e.g. 0.05) may be accepted and the label may be added to a copy of the input sequence before it is pushed onto the priority queue. This may be repeated until a set amount of sequences are completely generated.

In one or more embodiments, the ZF protein design model 1800 may be executed iteratively to incrementally determine the ZF protein sequence 1814. optionally wherein a single amino acid in the ZF protein sequence is determined per iteration of the ZF protein design model 1800.

In one or more embodiments, the determining the first embedding and the second embedding at the processor may further comprise receiving a first masked ZF protein subsequence and a second masked ZF protein subsequence; and the iterative execution of the ZF protein design model may comprise reducing the size of a mask of the first and second masked ZF protein subsequences.

In one or more embodiments, determining the candidate protein sequence 1814 may further comprise executing an iteration of a search algorithm based on the first masked ZF protein subsequence 1804a, the second masked ZF protein subsequence 1804b, and the protein design model 1800.

In one or more embodiments, the search algorithm may be the A* search algorithm. Executing the iteration of the search algorithm may comprise maintaining a priority queue of one or more partially masked ZF protein sequences and determining a probability of a top partially masked ZF protein sequence in the priority queue by processing the top partially masked sequence using the protein design model. In one or more embodiments, the probability of the top partially masked ZF protein sequence may be determined using the equation:

$${p}_{j} = {\sum }_{i = 1}^{j} log ( {p}_{i} ) + {\sum }_{j}^{12} log ( {p}^{*} )$$

which is a heuristic that approximates the maximum expected probability of a sequence that would be attained by predicting the remaining residues. pᵢ denotes the probability assigned to the prediction made at iteration i and j denotes the number of predicted residues. p* denotes the expected maximum probability that would be assigned by the network to later predictions. As set out in the Examples, this parameter may be tuned to move the search closer to a greedy search or a breadth first search.

In another embodiment, an alternative biased sampling approach using Monte Carlo sampling and/or temperature adjusted distributions may be used. As set out in the Examples. this approach may result in higher likelihood sequences. At every iteration, the probability of predicting an amino acid i at position j may be determined using the equation:

$${p ( {x}_{i , j} \vert n , {x}_{( k , m ) ∈ S} )}^{( T )} = \frac{{p ( {x}_{i , j} \vert n , {x}_{( k , m ) ∈ S} )}^{\frac{1}{T}}}{{\sum }_{a = 1}^{20} {\sum }_{b = 1}^{12} {p ( {x}_{a , b} \vert n , {x}_{( k , m ) ∈ S} )}^{\frac{1}{T}}}$$

wherein n denotes the input nucleotide sequence and S denotes the set of pairs of amino acids and positions that have already been predicted. T is an adjustable parameter that controls the bias of the distribution, which may be set to 0.6.

In one or more embodiments, the target nucleic acid sequence is made up of a pair of overlapping subsequences, each of which corresponds to the binding site for adjacent ZF helices in a ZF pair. In one embodiment, the first target nucleic acid subsequence 1802a and the second target nucleic acid subsequence 1802b are each 4 nucleotides in length. In one embodiment, the 5′ nucleotide of the first target nucleic acid subsequence and the 3′ nucleotide of the second target nucleic acid sequence overlap. For example, the overlap 1802c of first target nucleic acid subsequence 1802a and second target nucleic acid subsequence 1802b.

In one or more embodiments, the target nucleic acid sequence may comprise or consists of 7 nucleotides and the ZF protein sequence defines a ZF helix pair that binds to a target nucleic acid with the target nucleic acid sequence.

In one or more embodiments, the first ZF protein subsequence and/or second ZF protein subsequence each comprise a set of 6 amino acid residues. In one embodiment, the set of 6 amino acids define residues that contribute to the binding activity of a given ZF helix. In one or more embodiments, the set of 6 amino acids are not wholly contiguous, such as the residues at ZF motif positions −1, 1, 2, 3, 5 and 6 as shown in FIG. 18A.

Typically, a single ZF finger or helix will bind to 3-4 nucleotides. As set out in Example 2, the embodiments described herein may be used to determine extended ZF protein sequences comprising greater than two ZF helices that are useful for binding extended target nucleic acid sequences extended longer than e.g. 6-8 nucleotides.

In one or more embodiments, the ZF protein sequence may be an extended array comprising n helices and the target nucleic acid sequence has a target sequence of length 3n+1. For example, in one embodiment, the ZF protein sequence may define 3 zinc finger pairs targeting a nucleic acid sequence of 21 nucleotides.

In one or more embodiments, the ZF protein design model may be run n−1 times, one for each helix pair in the extended array of n helices.

In one or more embodiments, the first module and second module may be trained on single helix ZF specificity data, optionally wherein the single helix ZF specificity data comprises data on single ZF helix protein sequences that bind polynucleotides comprising or consisting of a target 4-mer.

In one or more embodiments, the third module may be trained on ZF helix-pair binding data, optionally wherein the ZF helix-pair binding data comprises data on ZF helix-pair sequences that bind polynucleotides comprising or consisting of a target 7-mer.

In another aspect, the methods described herein may further comprise synthesizing a polypeptide comprising an amino acid sequence based on a ZF protein sequence or a nucleic acid encoding said polypeptide. As set out in the Examples, use of the machine learning model described herein for determining a ZF protein sequence has been demonstrated to effectively target nucleic acid sequences and allow for the reprogramming of transcription factors. Polypeptides comprising a ZF protein sequence determined according to the methods described herein may readily be produced such as by using chemical synthesis techniques or by recombinant expression in a suitable host cell. In one embodiment, the polypeptides may be designed as transcriptional repressors. In another embodiment, the polypeptides may be designed as transcriptional activators.

Referring next to FIG. 19 there is shown a method diagram 1900 for reprogramming a biomolecule (e.g., a transcription factor such as a ZF protein, or an endonuclease to which a ZF scaffold can be fused) to bind a target nucleic acid sequence, in accordance with one or more embodiments

At 1902, a biomolecule is provided, or a nucleic acid encoding a biomolecule.

At 1904, the biomolecule or the nucleic acid encoding the biomolecule, is modified to bind the target nucleic acid sequence based on one or more ZF protein sequences. In one embodiment, the one or more ZF protein sequences are determined according to the method of FIG. 18A or FIG. 18B.

Referring next to FIG. 20 there is shown a method diagram 2000 for generating a model for determining a Zinc Finger (ZF) protein sequence for binding a target nucleic acid sequence, in accordance with one or more embodiments.

At 2002, a hierarchical machine learning model is provided comprising a first layer comprising a first module and a second module and a second layer comprising a third module, wherein embeddings from the first layer are fed into the second layer.

At 2004, the first module is trained based on single helix binding data. In one embodiment, the single helix binding data comprises data on single ZF helices that bind polynucleotides comprising a target 3-mer and one or more adjacent nucleotides, optionally at the 5′ end of the target 3-mer. In one embodiment, the single helix binding data comprises data on single ZF helices that bind a target 4-mer.

At 2006, the second module is trained based on single helix binding data. In one embodiment, the single helix binding data comprises data on single ZF helices that bind polynucleotides comprising a target 3-mer and one or more adjacent nucleotides, optionally at the 3′end of the target 3-mer. In one embodiment, the single helix binding data comprises data on single ZF helices that bind a target 4-mer.

At 2008, the hierarchical machine learning model is trained based on helix-pair binding data, wherein the helix pair binding data comprises data on ZF-helix pairs that bind polynucleotides comprising a target 6-mer. In one embodiment, the helix-pair binding data comprises data on ZF helix-pairs comprising single ZF helices that have been used to train the first and/or second modules.

In one or more embodiments, the first module, the second module, and the third module may comprise attention-based deep learning models.

In one or more embodiments, the first module, the second module, and the third module may comprise recurrent neural networks, optionally recurrent neural networks with long short-term memory. In one or more embodiments, first module, the second module, and the third module may comprise convolutional neural networks.

In one or more embodiments, the first module and the second module may comprise encoder models and the third module comprises a decoder model, optionally wherein the encoder models generate a high-dimensional representation for each DNA base in the target nucleic acid sequence and the decoder model generates predictions for each amino acid residue in the ZF protein sequence.

In one or more embodiments, the first module may generate a first embedding and the second module may generate a second embedding that are concatenated prior to being fed into the third module.

In one or more embodiments, the third module may comprise at least one self-attention layer and at least one feed forward layer.

In one or more embodiments the single helix binding data for training the first module and/or second module may comprise data generated from for example bacterial one-hybrid (B1H) selection libraries.

In one or more embodiments, the single helix binding data for training the first module and/or second module may comprise data on single ZF helices that bind polynucleotides comprising or consisting of a target 4-mer.

In one or more embodiments, the helix-pair binding data for training the third module may comprise data generated from bacterial one-hybrid (B1H) selection libraries.

In one or more embodiments, the helix-pair binding data for training the third module may comprise data on ZF-helix pairs that bind polynucleotides comprising or consisting of a target 7-mer.

In one or more embodiments, training one or more of the first module, the second module and the third module may comprise providing a target nucleotide sequence and sequence of partially masked ZF residues and evaluating a cross-entropy loss based on output probabilities.

Also provided in another aspect, is a reprogrammed biomolecule or a nucleic acid encoding said reprogrammed biomolecule, said reprogrammed biomolecule comprising the biomolecule and a ZF scaffold comprising one or more ZF protein sequences (e.g. designed ZF protein sequences) described herein, wherein the ZF scaffold comprising the one or more ZF protein sequences are fused to the biomolecule or replace a DNA binding segment thereof.

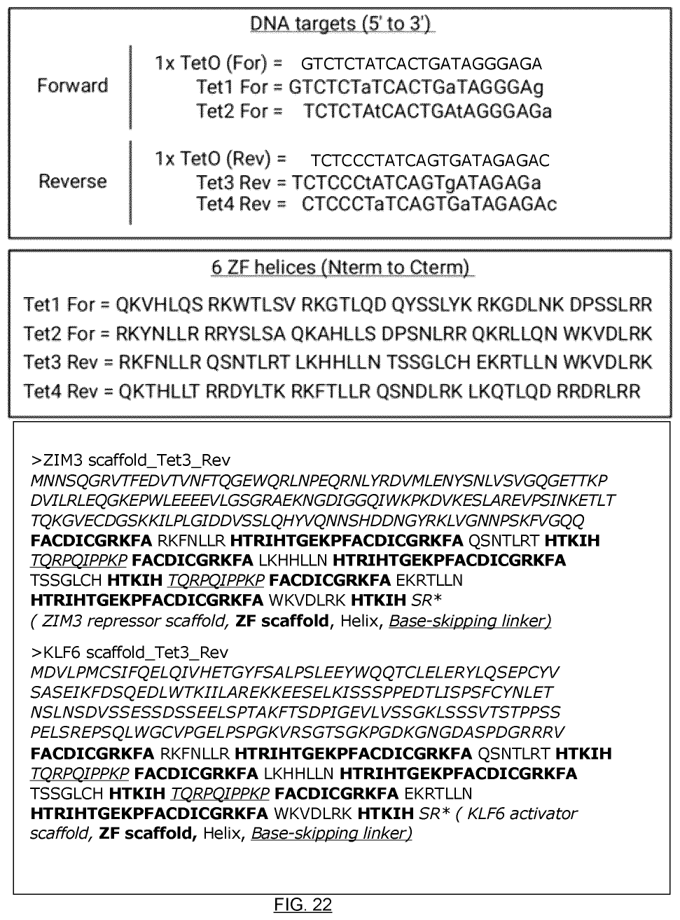

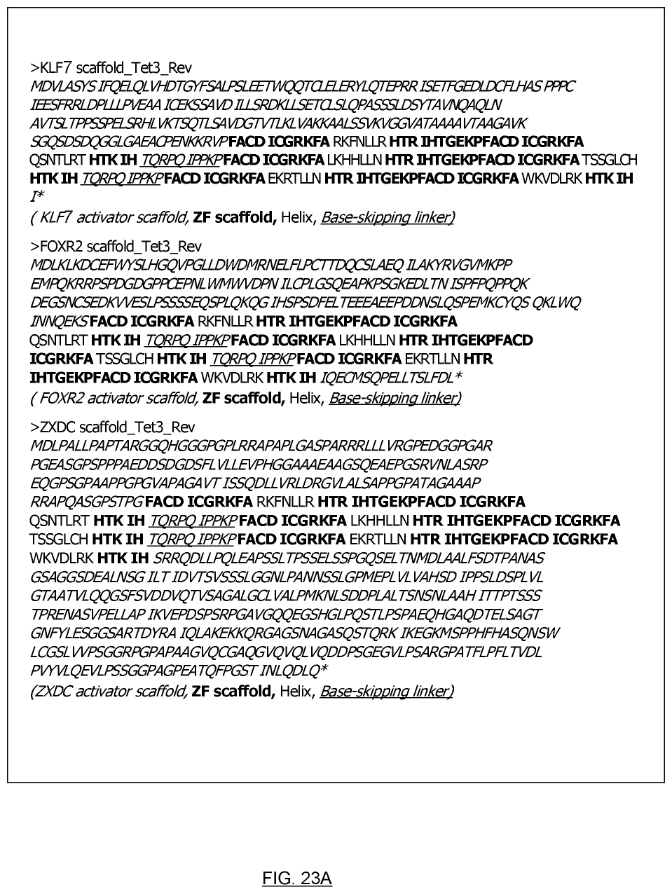

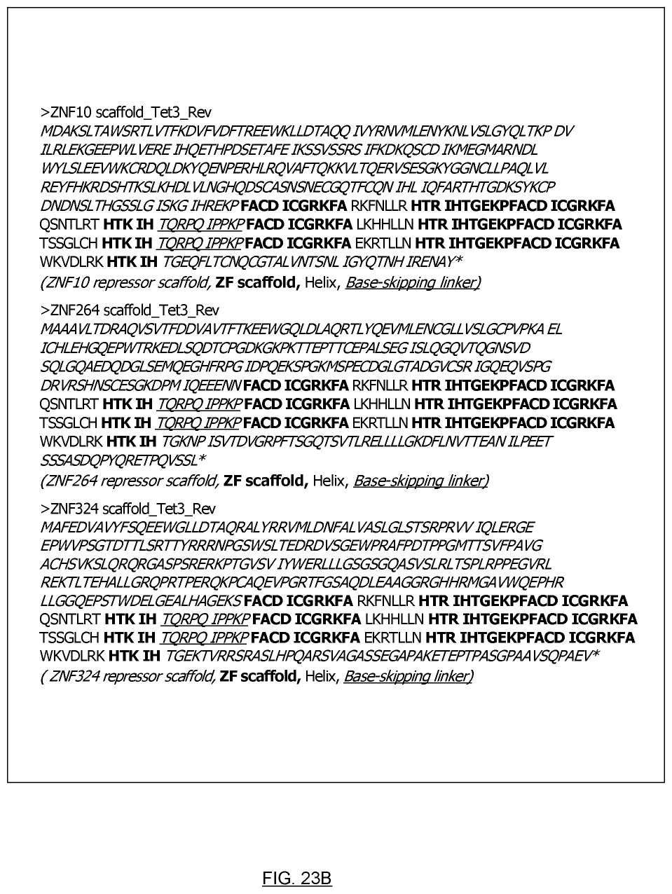

The biomolecule can be for example any protein that binds DNA or effector domain thereof. The ZF scaffold can be fused to the N-terminus of the biomolecule or the C terminus of the biomolecule. In one or more embodiments, the biomolecule is a transcription factor, or endonuclease or an effector domain of such a protein (e.g., activation domain, or endonuclease domain). The transcription factor or effector domain can for example be a ZnF protein or effector domain thereof wherein one or more of the native ZF protein sequences are replaced with one or more ZF protein sequences described herein (e.g., one or more designed ZnF protein sequences), optionally wherein a ZF scaffold replaces the ZnF protein DNA binding domain. The ZF scaffold can be in one or more embodiments. a ZF protein DNA binding domain, optionally based on ZiF268 as shown for example in FIG. 22 or 23. The designed ZF protein sequences (e.g. designed ZF protein helix sequences) can be used to replace or modify the natural ZF helices. The ZF scaffold can for example be fused to or introduced into a biomolecule such as ZIM3 (e.g. fused to the KRAB domain) or KLF6 (FIG. 22), KLF7, FOXR2, ZXDC, ZF10, ZNF264, or ZNF324 (FIG. 23) or any other ZF protein. Other ZF scaffolds, for example with at least 70%, 80%, 85%, 90%, 95%, or 98% identity to the ZF scaffold described herein can be used (e.g., where the identity calculation excludes the ZF helix sequences). The ZF scaffolds can replace for example any corresponding region in a ZF protein (e.g. DNA binding domain) or be added to any protein (e.g. transcription, nuclease etc) where sequence specific DNA binding would be advantageous. The ZF scaffold can comprise one or more linkers, linking one or more of ZF protein sequences. One or more of the designed ZF sequences, disclosed herein can be used.

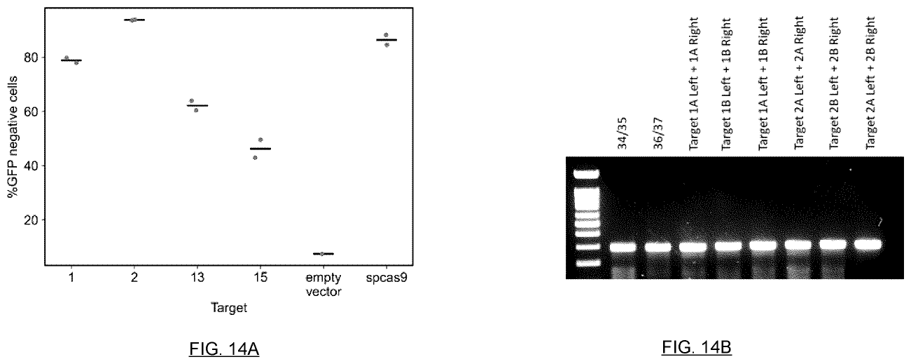

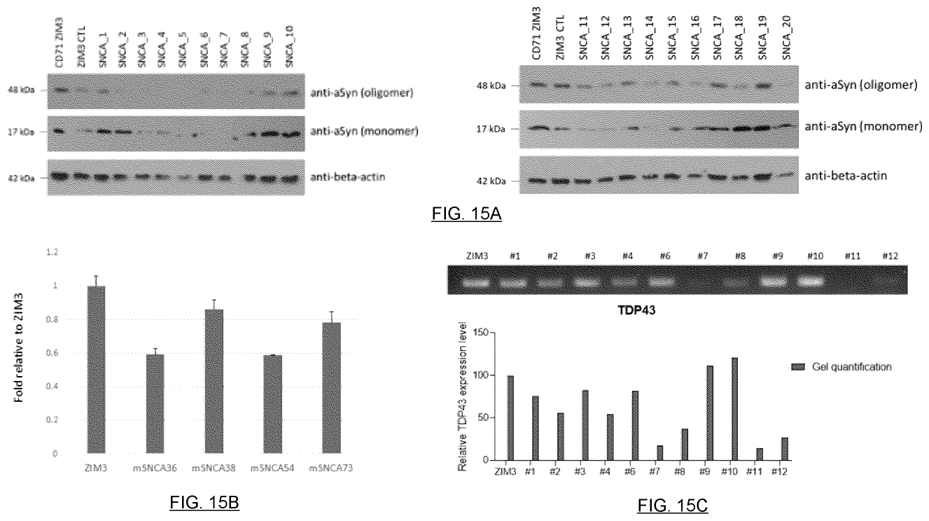

Natural ZnF proteins can have two or many zinc finger domains. One or more of the ZF domains in the natural protein can be replaced with the designed ZF protein sequences. The number of ZF protein sequences designed and incorporated into a ZF scaffold depends on fingers the length of the nucleic acid sequence to be targeted. In one or more embodiments, 2, 3, 4 5 or 6 of the ZF protein sequences are used. For example, the biomolecule can comprise 2, 3, 4 5 or 6 of the ZF protein sequences in Table 3. In one or more embodiments, the one or more ZF protein sequences are selected from the sequences described in Table 3. The ZF scaffold can comprise for example repeats of the ZF protein sequences In one or more embodiments, the one or more ZF protein sequences are the ordered sequences listed in Table 3. where for example at least 4, 5 or 6 of the helices are replaced in the ZF scaffold. As shown herein, nucleases and repressors that are reprogrammed biomolecules comprising the designed ZFs could be used to repress mouse SNCA (FIG. 15B) and to repress TDP43 (FIG. 15C).

In one or more embodiments, the biomolecule is a transcription factor. In one or more embodiments, the transcription factor is a ZF protein (e.g. comprising an activation domain, and a ZF DNA binding domain). In one or more embodiments. the biomolecule is a ZF nuclease. In one or more embodiments, the biomolecule is FOK1 or the effector domain thereof. As shown herein, the catalytic domain of FOK1 can be fused to a ZF scaffold comprising one or more ZF protein sequences that reprogram the fusion molecule so that it targets sequence in the GFP coding sequence. The one or more ZF protein sequences can be selected from Table 6, for example one or more of the ZF protein sequences shown for CCR5

In one or more embodiments, the biomolecule is a repressor.

The nucleic acid can for example be comprised in a vector, optionally operatively linked to a promoter.

The term “polypeptide” as used herein refers to a polymer consisting a number of amino acid residues bonded together in a chain. The polypeptide can form a part or the whole of a protein. The polypeptide may be arranged in a long, continuous and unbranched peptide chain. The polypeptide may also be arranged in a biologically functional way. The polypeptide may be folded into a specific three dimensional structure that confers it a defined activity. The term “polypeptide” as used herein is used interchangeably with the term “protein”.

The term “nucleic acid” or “nucleic acid molecule” as used herein refers to a sequence of nucleoside or nucleotide monomers consisting of naturally occurring bases, sugars and intersugar (backbone) linkages. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, Adeno Associated viral vectors and the like. The term “plasmid” as used herein generally refers to a construct of extrachromosomal genetic material, usually a circular DNA duplex, which can replicate independently of chromosomal DNA.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1: A Comprehensive Screen and Universal Zinc Finger Model Enable Transcription Factor Reprogramming

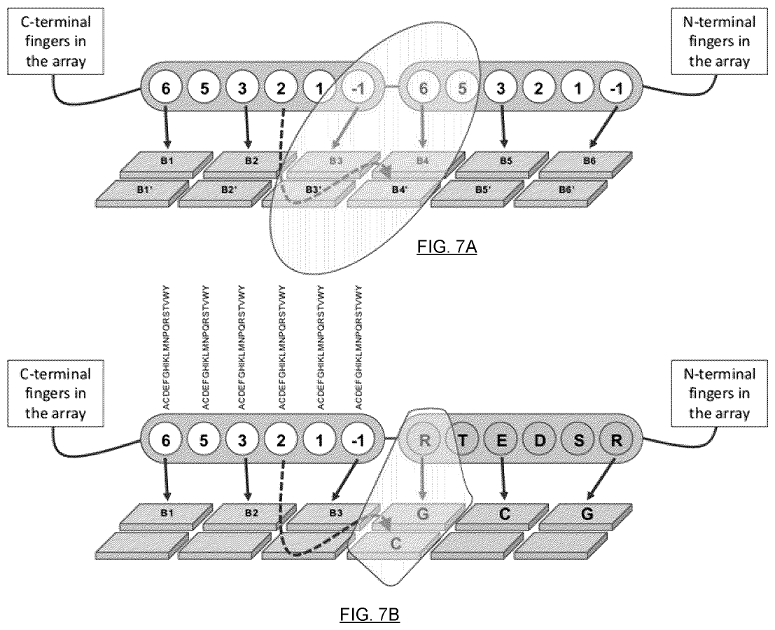

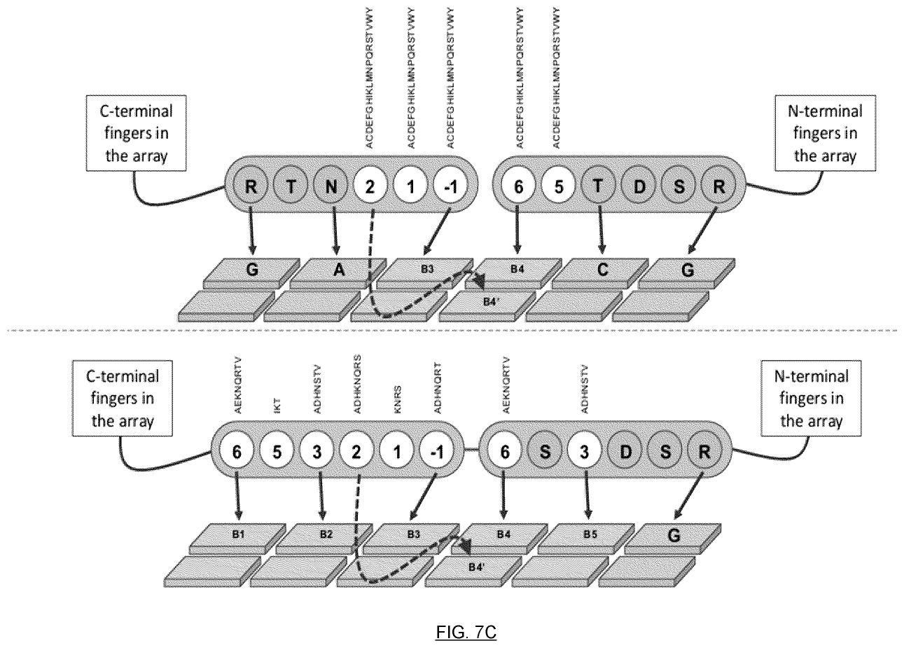

Two general approaches have been used to engineer ZFs with novel specificity (FIG. 7). The first focused on engineering one finger at a time by selecting functional variants from ZF libraries where the 6 base-specifying positions of the helix have been randomized (FIG. 7B). The second approach focused on the interface between adjacent ZFs of an array as the influence that adjacent fingers have on one another has been apparent since the first structures of ZFs bound to DNA were solved; this influence of course leads to combinatorially greater complexity, which is the main reason for the failure of previous attempts to build a code. While the first approach allows for a comprehensive screen of all amino acid combinations at the six critical positions of the ZF alpha helix(24, 26, 27, 29-32) it only samples these combinations in a single adjacent-finger context. As a result, only ZF strategies enabled by this initial single selection environment are available in subsequent rounds of selection or as the foundation of a ZF model. By contrast. the second approach captures the complexity of compatibility at the interface between ZFs(25, 28,33) (FIG. 7C). However, as combinatorial explosion quickly exceeds the maximum practical library size for any screening platform, incomplete randomization schemes and the sampling of a limited number of helical positions become necessary. It was therefore reasoned that the solution lies in a combined approach that uses multiple comprehensive libraries in a comprehensive set of interface environments. In other words, each library fully randomizes a single ZF helix in a unique interface environment. Multiple libraries and a diverse, comprehensive set of interface environments would produce broad portfolios of general and interface-specific ZF solutions. It was theorized that this interface-derived complexity would provide both the diversity necessary to generate compatible ZF pairs able to bind a wide range of DNA targets, as well as the depth of data required to support a model for ZF array design.

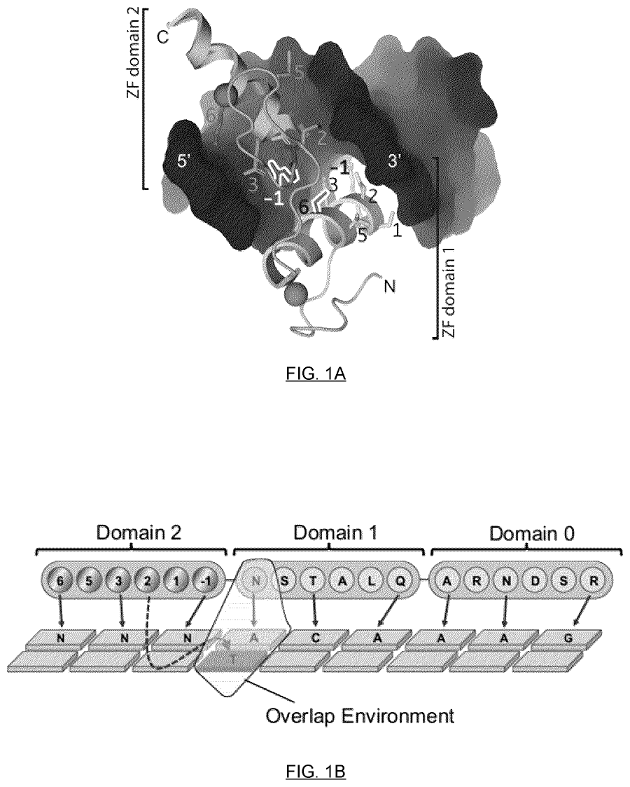

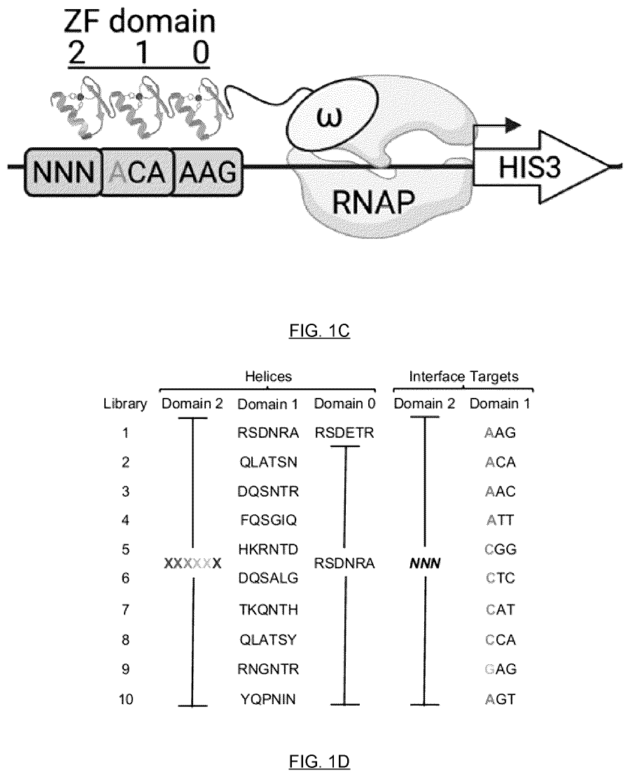

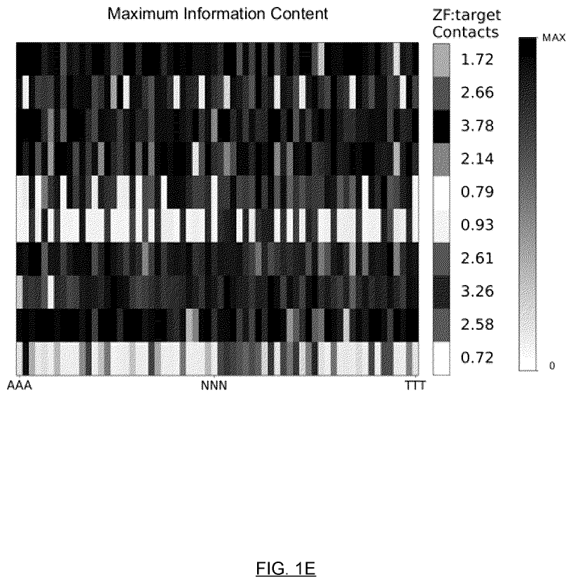

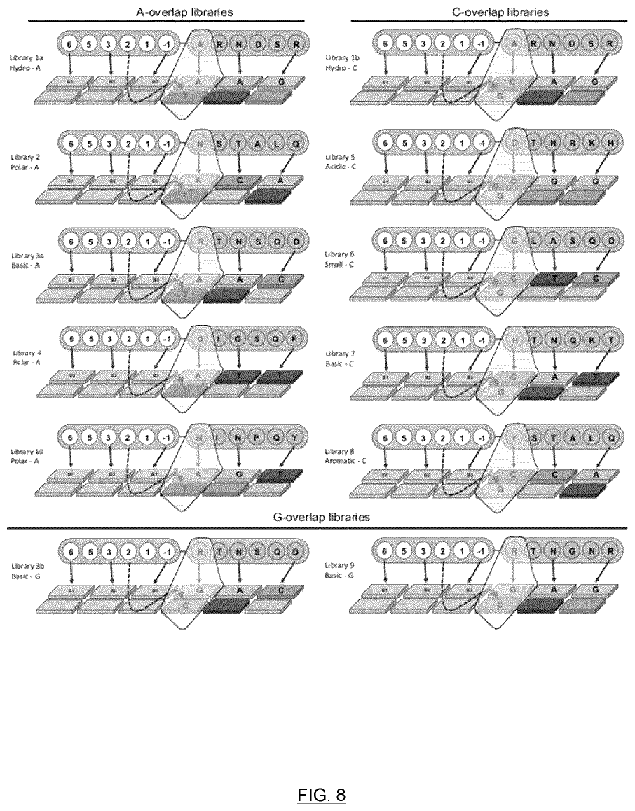

Multiple side chains from adjacent ZFs bind DNA in close proximity to one another; this is especially true at the binding site “overlap” where position 6 of an N-terminal helix can be within hydrogen bonding distance of the position −1 and 2 side chains of its C-terminal neighbor. At this position, the specificity of adjacent ZFs overlaps and in this way the N-terminal helix is presenting a specific interface environment to its C-terminal neighbor that is based on the side chain employed and the base specified (FIG. 1A and 1B). 10 ZF libraries were screened that each fully 20 randomized the six base-specifying positions of a C-terminal ZF helix using a bacterial hybrid assay (FIG. 1C). Each library puts the random C-terminal ZF helix in a different environment defined by the adjacent ZF helices. These libraries were screened across each of the 64 possible 3 base pair (bp) targets in independent selections to recover functional ZF helices. As the overlap environment should have the greatest adjacent finger influence on the ZF strategies selected in the screens, each library presents a unique interaction between the side chain at position 6 of the adjacent finger and the base it specifies at the overlap (FIG. 1D, FIG. 8 and Table 1). The majority of the libraries were designed to contact adenine or cytosine at the overlap in order to provide a contrast to the arginine-guanine contacts that have been presented at the overlap in the majority of prior ZF screens. In addition, two of the libraries can specify two different bases at the overlap (#1-A,C and #3-A,G). Therefore, two comprehensive screens were completed of these libraries, one screen with each base presented at the overlap. In total, over 49 billion protein-DNA interactions were screened from 10 libraries, across 12 sets of 64 selections per library, for 768 independent selections.

TABLE 1
List of libraries and interfaces tested.

| | | | | | Total | |
|---|---|---|---|---|---|---|
| | Domain | | | Total | helical | |
| Library | 1 | Overlap | Overlap | helices | “cores” | successful |
| # | helix | base | environment | recovered | recovered | selections |
| 1 | RSDNLRA | A | hydrophobic | 383952 | 27731 | 97% |
| 1b | RSDNLRA | C | hydrophobic | 580513 | 40882 | 98% |
| 2 | QLATLSN | A | polar | 294432 | 37005 | 86% |
| 3 | DQSNLTR | A | basic | 298638 | 34484 | 100% |
| 3b | DQSNLTR | G | basic | 735906 | 63649 | 92% |
| 4 | FQSGLIQ | A | polar | 398709 | 27434 | 97% |
| 5 | HKRNLTD | C | acidic | 264253 | 47964 | 78% |
| 6 | DQSALLG | C | small | 128306 | 36919 | 41% |
| 7 | TKQNLTH | C | basic | 494026 | 35203 | 100% |
| 8 | QLATLSY | C | aromatic | 293300 | 31362 | 97% |
| 9 | RNGNLTR | G | basic | 1089522 | 46578 | 97% |
| 10 | YQPNLIN | A | polar | 620359 | 88293 | 39% |

Table. All libraries screened in this Example are listed. The helical residues for the zinc finger adjacent to the library (domain 1 as shown in FIG. 1) are shown for each library. The residue presented at the interface (underlined-last amino acid of domain 1 helix), the overlap base, and the biophysical category of this side chain is noted. Helical enrichment numbers and selection success is also listed. Library 1a and 1b are the same library 5 using a different base at the overlap position. The same is true for library 3a and 3b. These are referred to as libraries 1(A), 1(C), 3(A), and 3(G) to indicated what overlap base was used in the selections and explains why 10 libraries are presented but 12 screens were completed.

From these screens global and target-specific differences were found between these library contexts, indicative of the strength of the constraint that each context puts upon the C-terminal ZF. The total number of selected helices ranged from 128,000 to over 1 million helices per library screened (Table 1). MUSI(34), a method designed to identify multiple specificities in such data, was used to define ZF clusters for each library selection and to identify selections with low information content due to failed enrichment. The presence of at least one cluster that demonstrates low entropy was used as a definition of selection success (Table 1). To provide a quantitative comparison across all selections the maximum information content at a single helical position in any recovered cluster was used, reasoning that a successful selection should produce clusters where at least one position has been strongly selected for (FIG. 1E). From this analysis it was found that libraries were able to enrich helices in 39% to 100% of the 3 bp target selections (Table 1). In fact, ZF strategies were enriched in over 85% of the 3 bp target selections for 9 of the library screens. In addition, ZF strategies were enriched in at least 8 different library screens for each of the 64 3 bp targets, demonstrating the ability of ZFs to bind any target in a wide range of adjacent finger environments. Also note that at least one library that bound either A, C, or G at the overlap successfully enriched helices in over 95% of the selections (libraries 1-A overlap, 7-C overlap, and 9-G overlap), suggesting that ZF strategies exist in a wide variety of contexts independent of overlap base, further underlining the flexibility of the ZF scaffold. Libraries 6 (C overlap) and 10 (A overlap) were found to be the least successful libraries (Table 1); molecular dynamic simulations suggest that the number of contacts between the adjacent finger (domain 1 in FIG. 1D) employed in each library and the DNA it specifies correlates with global library success, indicating that higher affinity of the neighboring finger enables more ZF strategies (FIG. 1E). Hence, ZF function is significantly impacted by the adjacent finger interaction, while viable ZF binding strategies exist for each overlap base.

### G-Rich Binding Modularity and Promiscuity

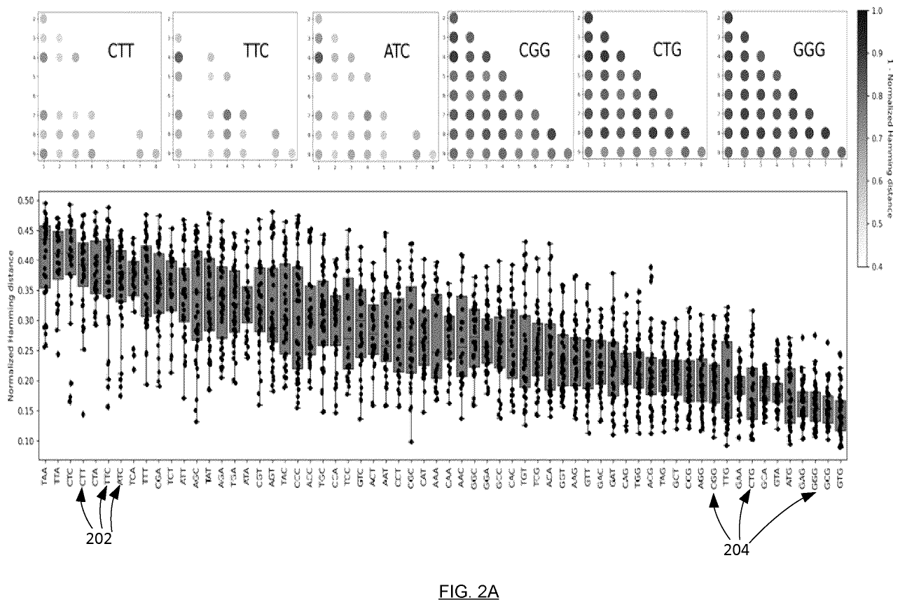

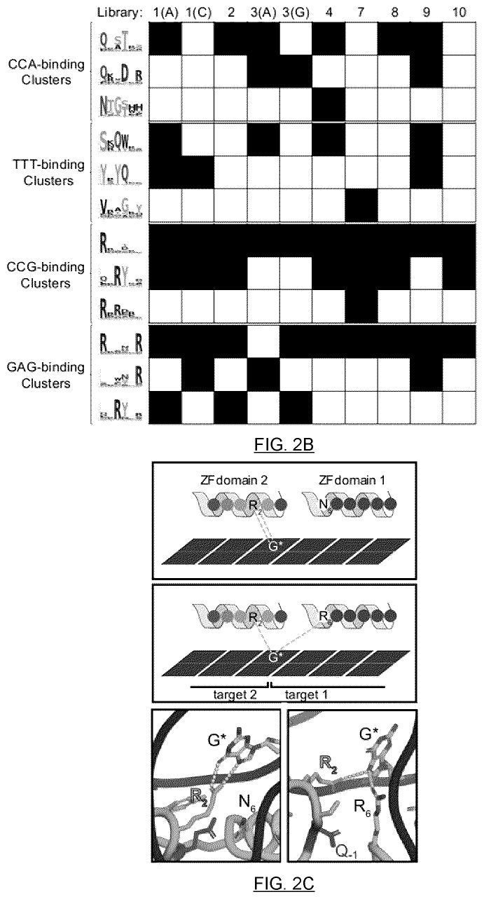

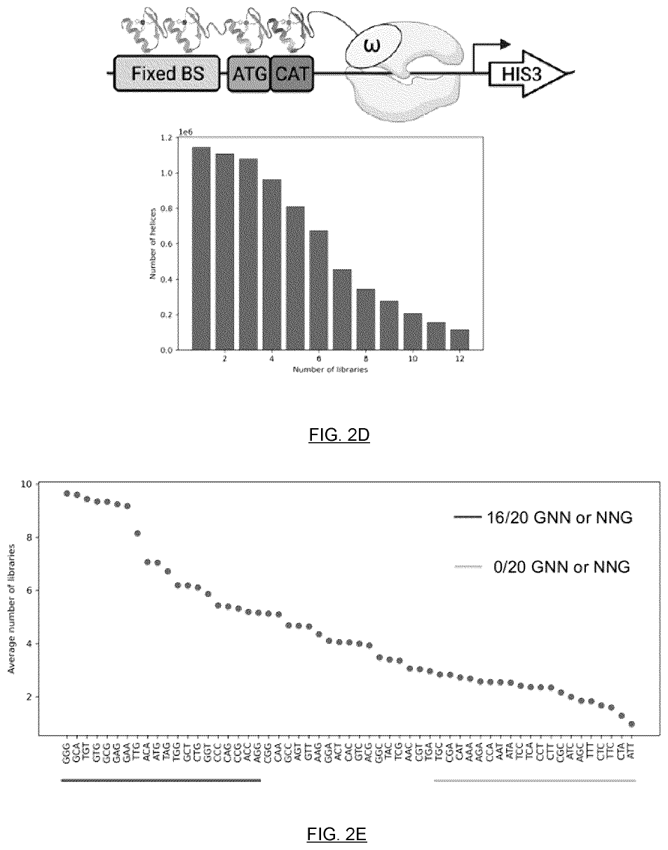

Since the majority of prior ZF selections have been carried out with an arginine-guanine contact presented at the overlap, it was reasoned that libraries that present adenine and cytosine contacts would enrich novel helical strategies. To measure these differences on a global scale mean Hamming distance was first calculated between the helices enriched to bind each target across all libraries. Next, the normalized Hamming distance for all targets were compared to compare library differences. While there are general trends that libraries that employ the same overlap base are more similar, the most striking difference is found when comparing libraries with adenine and cytosine at the overlap to the two libraries that displayed an arginine-guanine contact at the overlap. The arginine-guanine contact libraries are more similar to each other than any of the other libraries screened. Interestingly, a comparison of target selection hamming distances across all libraries shows G-rich binding is less influenced by the library context. This suggests that G-rich binding is more modular as these helices appear less dependent on the adjacent finger interaction (FIG. 2A). However, this independence in binding could lead to more promiscuity. To address this possibility helices recovered in each 3 bp target selection were considered and how frequently these helices are recovered in other target selections was calculated. The 15 targets with the greatest target selection entropy (i.e., are recovered in the most other selections) all have a G at the GNN or NNG positions where arginine's are the dominant amino acid enriched at the corresponding positions 6 and −1, respectively. Conversely, none of the 13 targets with the lowest target selection entropy have a G at these positions. These results demonstrate that helices that bind a G at either the first or third position of a binding site are more likely to be promiscuous ZFs. This could help explain why prior selections have largely led to a G-rich bias in ZFs that have been successfully engineered or assembled as modules, with these modules also likely tending towards more off-target binding.

### General and Specialized Binding Strategies

Global differences between library environments were assayed by the success of selections across targets as well as the mean Hamming distances. To investigate more specific differences, such as the types of binding strategies enabled by one library environment versus another, the clusters generated by MUSI for each target site selection were compared. For most targets general strategies were found that are common to several successful library selections. Specialized strategies that are recovered in a small number of selections and in some cases, only recovered with a single library environment (FIG. 2B) were also observed. Recovery of helical strategies in one library versus another has been shown to be predictive of activity only in the recovered contexts, confirming that these differences are not due to sampling influences(35). In addition, as these differences suggest structural influences at the overlap, whether the presence of a cluster in various library environments might suggest physical influences was considered. Interestingly, in most NCG selections a cluster of “QxRYxx” helices (see CCG in FIG. 2B) was found. However, this cluster is not recovered in libraries that presented an arginine from the adjacent finger at the overlap. Molecular dynamics simulations suggest that this is due to a potential competition between the arginine at position 6 of the adjacent finger and position 2 of the selected finger (FIG. 2C).

The data reported here demonstrate global and specific differences in ZF function influenced by the adjacent finger environment. While this data represents the largest screen of ZF function to date, it is still a relatively small number of the potential overlap influences. To test how greater variability at the interface might influence compatibility 200 two-finger libraries were created by assembling pools of helices selected to bind each 3 bp half-site of a 6 bp target. Compatible pairs of ZFs were selected from these libraries and how many starting library environments the helices were enriched from was analyzed. Most helices enriched in these compatibility assays were only recovered in a minority of the library environments (FIG. 2D). This suggests that despite the fact that all of these helices were pre-selected to bind each half site, only a fraction are enriched in these new environments. Interestingly, when the compatible helices by target selection was plotted and assayed the number of primary libraries they were recovered in, it was found that G-binding ZFs recovered in the 2-finger selections originate in a large number of the primary libraries while compatible ZFs recovered to bind G-poor targets originate in a small number of selections (FIG. 2E). Together, these results demonstrate that, even for a more comprehensive set of presented environments, the interface has a large influence on ZF function and that G-rich binding helices tend to be more modular and promiscuous. Importantly, the data from these two-finger library selections offers crucial insight into the pairwise compatibility of individually functional ZFs.

### A Hierarchical Attention-Based Neural Network Integrates Interface-Derived Selection Data

Despite considerable effort, all previous attempts at generating a general ZF design code have failed. Given the unprecedented depth of the screening data, a novel and unique model was developed that explicitly addresses these neighbor influences. In particular, the model separately makes use of the single-finger library selections that comprehensively describe single-finger specificity in a variety of neighbor finger contexts and the pair selections that show which ZFs are compatible with each other as neighbors. This information is by nature hierarchical and to make optimal use of it. a novel neural network architecture was developed that implements attention modules in a hierarchical manner (FIG. 3A).

The data reported here demonstrate global and specific differences in ZF function influenced by the adjacent finger environment. While this data represents the largest screen of ZF function to date, it is still a relatively small number of the potential overlap influences. To test how greater variability at the interface might influence compatibility 200 two-finger libraries were created by assembling pools of helices selected to bind each 3 bp half-site of a 6 bp target. Compatible pairs of ZFs were selected from these libraries and how many starting library environments the helices were enriched from was analyzed. Most helices enriched in these compatibility assays were only recovered in a minority of the library environments (FIG. 2D). This suggests that despite the fact that all of these helices were pre-selected to bind each half site, only a fraction are enriched in these new environments. Interestingly, when the compatible helices by target selection was plotted and assayed the number of primary libraries they were recovered in, it was found that G-binding ZFs recovered in the 2-finger selections originate in a large number of the primary libraries while compatible ZFs recovered to bind G-poor targets originate in a small number of selections (FIG. 2E). Together, these results demonstrate that, even for a more comprehensive set of presented environments, the interface has a large influence on ZF function and that G-rich binding helices tend to be more modular and promiscuous. Importantly, the data from these two-finger library selections offers crucial insight into the pairwise compatibility of individually functional ZFs.

### A Hierarchical Attention-Based Neural Network Integrates Interface-Derived Selection Data

Despite considerable effort, all previous attempts at generating a general ZF design code have failed. Given the unprecedented depth of the screening data, a novel and unique model was developed that explicitly addresses these neighbor influences. In particular, the model separately makes use of the single-finger library selections that comprehensively describe single-finger specificity in a variety of neighbor finger contexts and the pair selections that show which ZFs are compatible with each other as neighbors. This information is by nature hierarchical and to make optimal use of it, a novel neural network architecture was developed that implements attention modules in a hierarchical manner (FIG. 3).

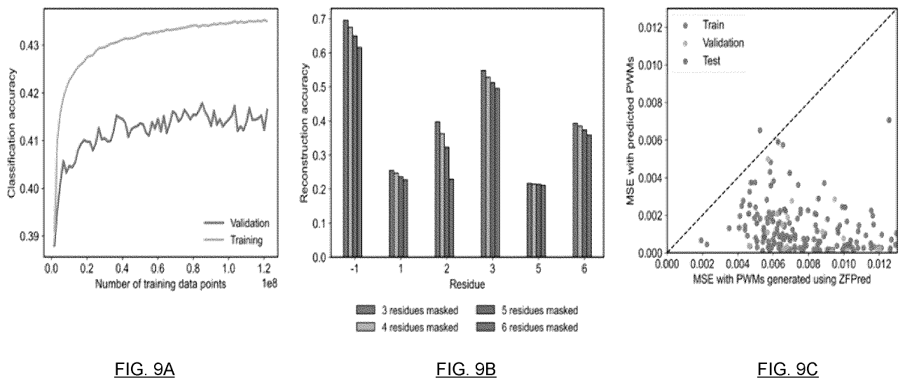

The first layer of this hierarchical architecture contains two modules that are trained on the single-finger selection data sampling a wide range of influences at the interface where adjacent finger specificity can overlap (FIG. 3). The single-helix modules generalize to unseen sequences; interestingly, residue-nucleotide relationships are captured in the attention values (FIG. 9). The residue embeddings from the bottom layers are then fed into a top module which is trained on the two-helix selection data (FIG. 3). This is akin to the experimental procedure of taking the selection pools from the single finger selections and performing two-finger selections on them. In effect, the bottom modules design functional single ZFs (for a given neighbor environment), while the top module assembles compatible ZF pairs.

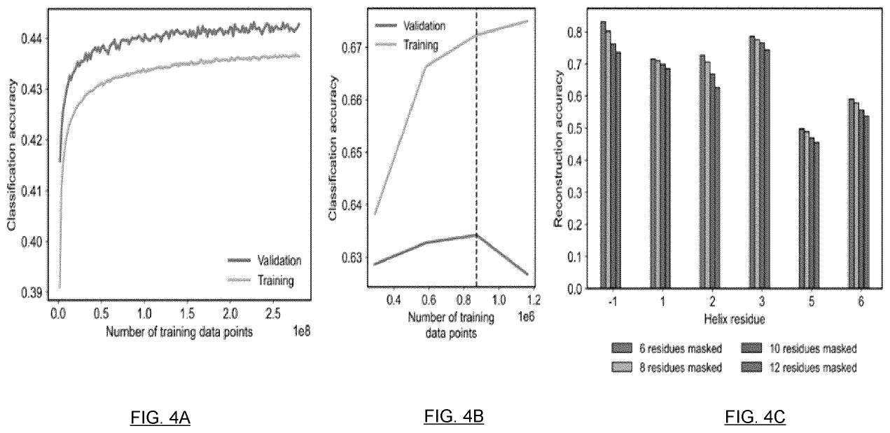

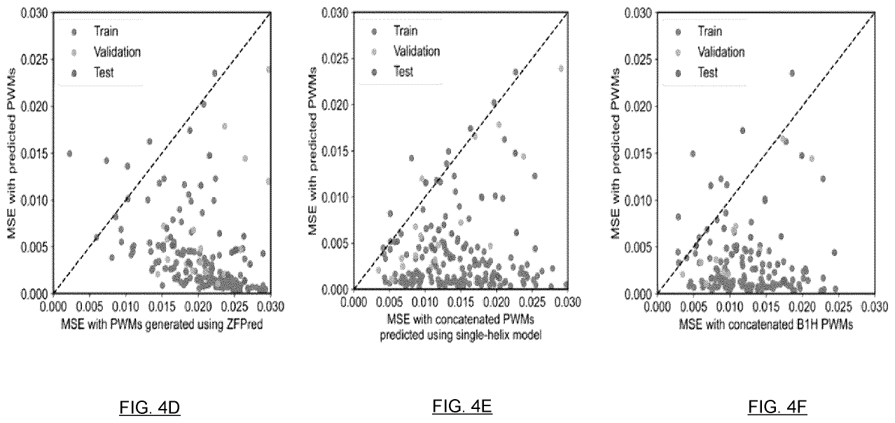

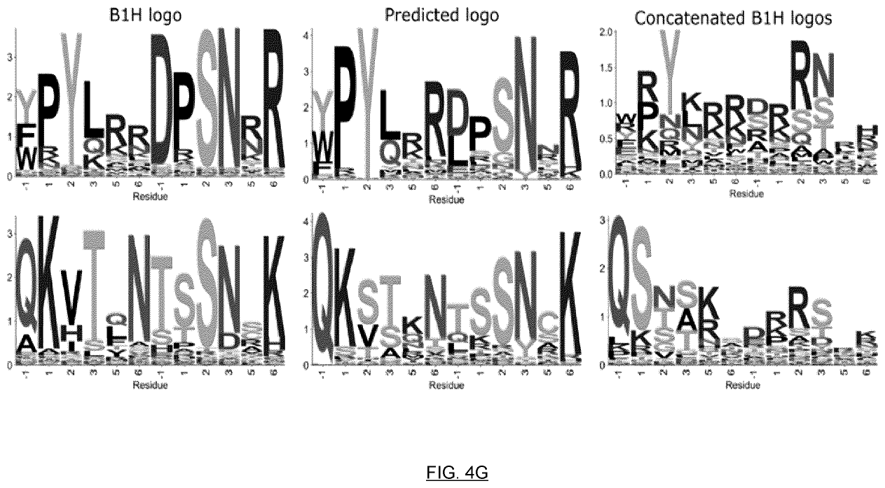

The overall model retains a traditional encoder-decoder architecture: An encoder generates a high-dimensional representation for each DNA base, a decoder then generates predictions for each residue in a ZF helix using self-attention layers and attention layers that relate the nucleotide bases to the helical residues. To train the model, the nucleotide target as well as a partially masked ZF sequence was provided and the cross-entropy loss given input data was evaluated (see Materials and Methods below). A reconstruction accuracy (sequence identity to the six masked residues) of 0.62 and 0.69 was achieved on the validation and test data respectively; some positions (such as “−1”) that are strong determinants of binding specificity having higher reconstruction accuracies (FIG. 4A-C). Overall, as some variability in the 12 residues is allowable while retaining the ability to bind a target sequence, 0.62-0.69 reconstruction accuracy can be considered quite high (See FIG. 4C).

### ZFDesign Accurately Captures Two Helix ZF Specificity

The method described herein (ZFDesign) generates sequences in an incremental fashion: Starting from an empty sequence, the model is run once for each amino acid in the ZF helix pair. At each iteration an amino acid is predicted and this prediction is provided as context in subsequent iterations. For optimal sequence generation both an A*-based sampling methodology(36), as well as a temperature-dependent sampling procedure(37) were adapted. It was sought to compare ZFDesign to a baseline, but no previous model has explicitly attempted to do full ZF-array design for a given target, with only a few collections of ZFs available. However, previous models that were designed to capture ZF binding specificity exist and can be adapted to design ZFs for given targets; ZFpred, a recently developed method that outperformed previous models(35) was used. Both ZFDesign and ZFpred were then used to generate ZF sequences to target 6-mers from our test dataset. As alternative baseline comparisons, the single-finger models (e.g., only the bottom module in FIG. 3) were first used to generate ZF sequences for each DNA 3-mer and concatenated. In a similar fashion, sequences were taken directly from each 3-mer B1H selection and concatenated, which is akin to previous methods of simply concatenating pre-existing collections of fingers as modules. All these three methods performed noticeably worse than the hierarchical model described herein (See FIG. 4D-F). When directly comparing representative sequence logos of the sequences generated, ZFDesign produces logos that broadly capture the ones from the B1H two-helix selections, whereas the concatenated logos from the one-helix selections are noticeably different (See FIG. 4G), underlining the fact that ZFDesign captures inter-helix relationships that are absent from the single-helix selections.

### ZFDesign, Zinc Finger Nucleases and Genomic Labeling

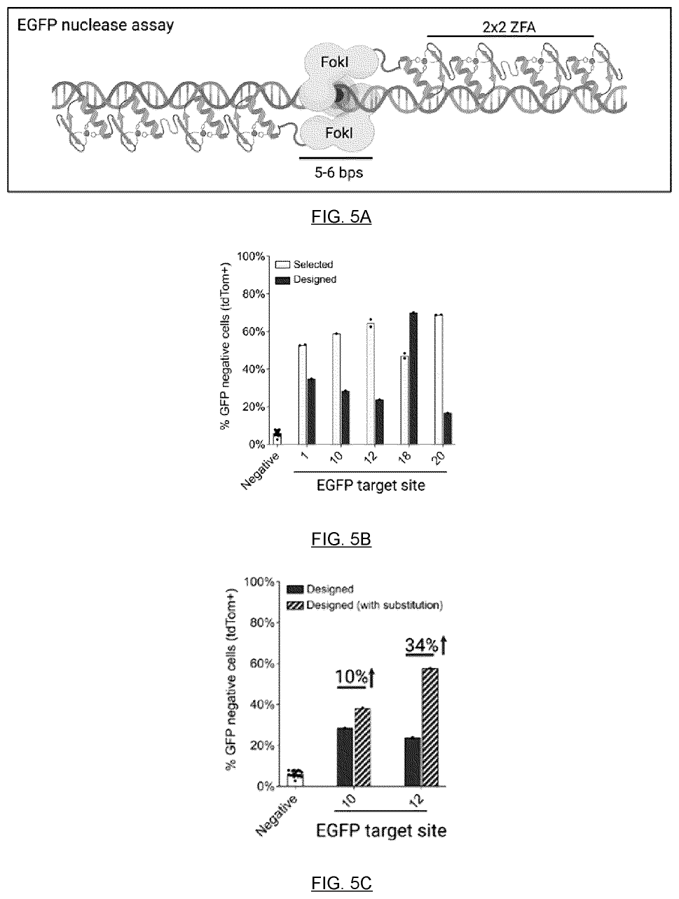

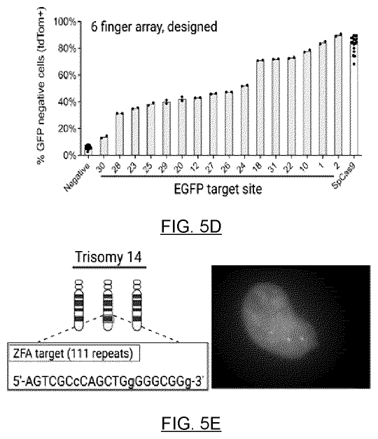

To validate ZFDesign a GFP-disruption assay was used in a U20S cell line that has been used to approximate nuclease activity for ZFNs(38), TALENs(39), and spCas9(40) as indels in the coding sequence of GFP lead to frameshifts and loss of fluorescence. For each ZFN, two ZF arrays were designed as ZFNs require dimerization of the Fok1 catalytic domain presented as C-terminal fusions from each ZF array in a tail-to-tail orientation (FIG. 5A). The arrays use a longer linker between two-finger modules to enable independent binding as the linker allows a base to be skipped between the binding sites for each two-finger module(41). The DNA targets for the two-finger selections detailed above had been specifically chosen to accommodate targets in the GFP coding sequence. Therefore, for each target ZFNs that use 4ZFs per monomer (8 per ZFN) were first assembled based on the most frequent pairs recovered in the corresponding 2-finger selections. Next, 5 ZFNs were designed that also use 4 ZFs per monomer to compare to the B1H selected ZFs that bind the same targets. All of the designed ZFNs are functional above background but 4 of the 5 demonstrated decreased activity relative to the selected arrays (FIG. 5B). However, the substitution of single modules can significantly increase activity (FIG. 5C) demonstrating the stringency of the assay as a single weak module can have a large impact on the overall function. Nevertheless, as these designs were functional on all targets, and longer arrays have overcome the presence of weak modules(42), 16 ZFNs were designed and tested that use 6 ZFs per monomer (12 per ZFN). All 16 were found to be functional with a mean 53.6% loss of fluorescence (FIG. 5D). Finally, to determine if 6-fingers are sufficient for monomeric binding, 6-finger arrays were designed to label a genomic locus as a GFP fusion. Many copies of GFP are necessary to visualize punctate GFP expression, the array was designed to bind a repetitive sequence on chromosome 14, which appears in trisomy in Hek293T cells. 3 points of GFP were observed by live cell imaging (FIG. 5E). These results suggest that

ZFdesign consistently produces highly functional ZF arrays and that 6 or more fingers routinely produce strong on-target activity in the human genome.

### Seamless Reprogramming of Human Transcription Factors

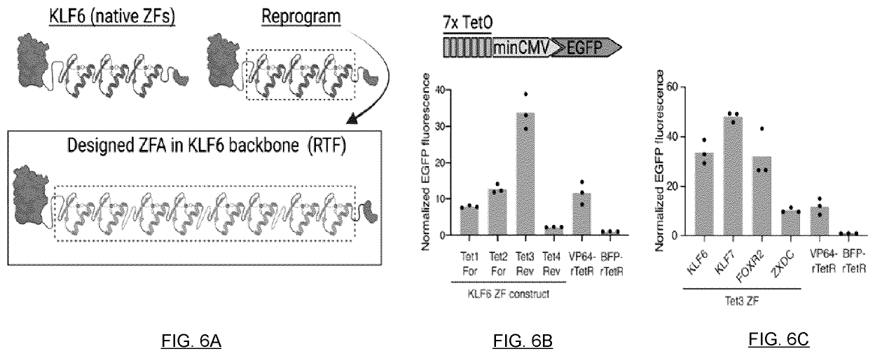

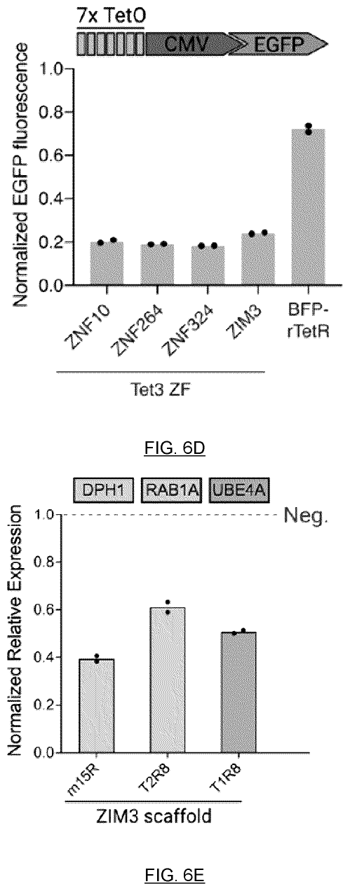

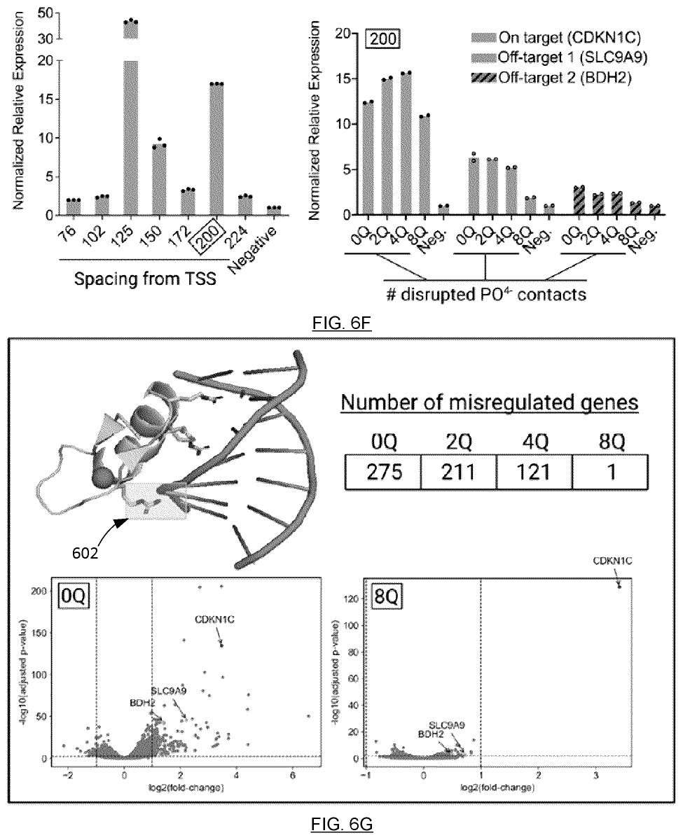

To avoid the presentation of effector domains out of their natural context, it was reasoned that ZF domains in human TFs could be seamlessly replaced with designed ZFs. This approach presents the designed ZFs in the exact context that ZFs would occur naturally in the parent protein. Such Reprogrammed Transcription Factors (RTFs) maximize secondary interactions of the TF, avoid the use of foreign effector domains, and enable research focused on the precise investigation of TF binding events (FIG. 6A). As potential therapeutics they present maximally native-like human proteins with correspondingly low immunogenicity risk. KLF6 was chosen as the activation scaffold. To test the activity of the KLF6 architecture four ZF arrays were designed to bind the TetO sequence on either the forward or reverse strand. KLF6's ZFs were replaced with these designed ZF arrays and these RTFs were expressed in a HEK293T reporter cell line that drives GFP expression with a minimal promoter (FIG. 6B). Three of the four designs activate at a similar or greater level than rTetR-VP64 with one array nearly tripling the activation level. To confirm that this RTF approach for activation was not restricted to the KLF6 protein, the DBDs of 3 other activating TFs (KLF7, FoxR2, and ZXDC) were replaced with the Tet3 ZF array (FIG. 6C). All of these RTFs activate the reporter as well or better than the rTetR-VP64 control including the FoxR2 RTF where its natural forkhead DBD was replaced with the ZF array.

To create RTFs that repress target genes ZIM3 was used as the TF scaffold as ZIM3's KRAB domain has proven a potent repressor as an isolated SpCas9 fusion(43). ZIM3's ZFs were replaced with the series of ZF arrays designed to bind the TetO sequence as described for KLF6. These ZIM3 RTFs were expressed in a HEK293T cell line with a GFP reporter driven by a constitutive promoter. Three of the four ZF arrays repress GFP expression relative to controls with the Tet3 array out performing dCas9. Next. the ZFs of three other KRAB-containing proteins (ZNF10, ZNF2.64, and ZNF324) were replaced with the Tet3 ZF array. In all cases similar levels of repression were observed (FIG. 6D). Interestingly, the Kox1 KRAB domain (ZNF10) provides less repression potential than the Zim3 KRAB domain when expressed as an isolated spCas9 fusion domain(43) but their activity is similar when expressed here as RTFs, suggesting that the presentation context can have a large impact on the potency of these domains.

To test the regulatory potential of endogenous genes with RTFs, the ZIM3architecture was applied to repress 3 endogenous targets (DPH1, Rab1a, and UEB4A) and 4 arrays were designed each to bind sequences close to the transcriptional start site (TSS) of each gene. To maximize the likelihood of function, these and all following ZF arrays were designed to use 8-fingers. HEK293T's were nucleofected with the RTFs and expression levels assayed by RT-qPCR. For each target gene at least one construct reduced expression levels significantly (FIG. 6E). To activate an endogenous target, KLF6 was reprogrammed with a series of arrays designed to bind a 150 bp region upstream of the TSS in the CDKN1C promoter. All 7 RTFs increased the expression of CDKN1C with 3 of the 7 by 9 to 43-fold (FIG. 6F).

### Genome-Wide Regulatory Activity of Reprogrammed Transcription Factors

ZFDesign enables the reprogramming of TFs for either activation or repression. To test the precision of the regulation RNA-seq was used to quantify the on and off-target regulation of the RTFs. The 4 most potent KLF6 RTF regulators of CDKN1C, #125, 150, 172, and 200 were investigated (see FIG. 6F). In all cases but #172 CDKN1C was one of the most upregulated genes. However, between 268 to 1173 off-target genes were also activated. Since KLF6 is a human TF, it was questioned whether off-target activity is due to secondary interactions of the TF and not the ZF arrays. KLF6 was therefore tested without any ZFs as well as the 4 ZF arrays as full KLF6 RTFs, as fusions with the KLF6 truncated transactivation domain, and as fusions with VP64. RNA-seq on each of these constructs indicates that off-target activity is primarily dictated by the ZF arrays.

The specificity of ZF arrays can be impacted by target content and affinity. As noted, G-rich binding tends to be more promiscuous. Consistent with this observation, the CDKN1C target with the lowest G-content (#200) also led to the least number of off-target events. In addition to minimizing target G-content, ZF specificity can be improved by reducing the nonspecific affinity provided by contacts made between each ZF and the phosphate backbone(44, 45) (FIG. 6G). This puts more pressure on the base-specifying interaction of each helix to provide the binding affinity necessary for function. Mutant versions of CDKNIC RTF #200 were created that replace either 2, 4, or 8 of the phosphate-contacting arginines with glutamines. The impact of these mutations by qPCR both on-target and at two off-target loci upregulated in the RNA-seq screens was compared (FIG. 6F, right). The expression of these off-target genes is reduced by up to 70 or 55%, respectively, as the number of phosphate-contacting modifications was increased while the on-target activity is only reduced by 12%. Next. RNA-seq demonstrates the number of off-targets is decreased with the number of modifications and that only CDKN1C is upregulated with the full 8 arginine to glutamine modifications, thus providing single target resolution. Taking the same approach with the G-rich binding #125 cut the number of off-targets in half but elimination of off-target activity will likely require the design of ZF-arrays that use alternative binding strategies for G-rich targets.

### Discussion

Described herein is a novel hierarchical attention-based Al model (termed ZFDesign) trained on comprehensive screens of ZF-DNA interactions that consider the influence of multiple adjacent finger environments. ZFDesign captures these influences to provide the first general design model for ZF arrays. By contrast, previous efforts produced incomplete collections of ZF modules that often fail out of context and produce low on-target activity. Conversely, the model described herein consistently produced ZF arrays across a wide range of targets with high efficacy as nucleases, repressors, and activators. Thus,

ZFDesign represents a significant advance as the design of ZFs for any given target is now available at the push of a button to study a myriad of academic and therapeutic applications with the advantages of small size and low immunogenicity.

The model was trained on two-finger selections that sampled less than 5% of the possible 6bp targets and single finger selections have not sampled T at the overlap positions. Therefore, more confidence may be assumed for domains that bind ‘V’ at the overlap. Further, each ZF design represents a new protein and potentially a corresponding set of off-target interactions. Nevertheless, the modification of nonspecific affinity has been demonstrated to improve specificity for ZF designs, even to single-target resolution.

### Materials and Methods

### Library Builds

Primary zinc finger libraries: All primary ZF libraries were built as previous described(35, 46) and detailed below. To provide templates for PCR, gBlocks were ordered from IDT that coded for the finger 0 and finger 1 domains of each library (FIG. 8, and see FIG. 1 for numbering of domains). The critical differences that distinguish each library from one another is that they each place a different environment at the interface between domain 1and the library domain 2. These libraries include five domain 1 interactions that bind A at the interface, five that bind C at the interface, and two that bind G. These libraries use side chains at the interface with a range of biochemical properties to interact with the overlap base (basic, acidic, polar, aromatic, and hydrophobic interactions). Together, the biochemical property of the side chain at position 6 of domain 1 and the base it specifies at the overlap position represent the unique interface environment offered by each library. Next, an oligonucleotide was designed with degeneracy (NNS) at the codon positions corresponding to the six critical residue positions of the ZF domain 2 alpha helix. This oligo was used for all library builds, only the template gBlock, and therefore the 0 and 1 domains, are changed. PCR was used to generate the library insert, amplifying from the library-specific gBlock template with the library oligonucleotide paired with a downstream oligonucleotide used to capture the full 3-finger insert. For each library, PCR reactions were run in 96-well plate format and pooled. The PCR products were digested with Kpn1 and XbaI and ligated into 15 μg of digested B1H expression vector. Ligations were run over night at 16° C., ethanol precipitated, and resuspended in 15 μl of 10 mM Tris-Cl, pH 8.5. The ligation was electroporated into 15 aliquotes of electrocompetent US0 cells and recovered in 1 L of SOC. One-hour post electroporation, 200 μl of the culture was titered in 10-fold serial dilution on Carbenicillin plates to determine library size. To select for transformants, carbenicillin was then added to the culture at this point and grown to mid-log. The library DNA was then recovered by Qiagen maxiprep. Library sizes ranged from 1-3×10⁹. This approach has been shown to consistently produce libraries with diversities that approximate random(46).

2-finger libraries: Second round selections were used to select compatible pairs from pre-selected ZF pools generated in the primary ZF library selections. Recovered plasmid DNA was pooled from the primary single-finger screens on a binding site basis, resulting in a pool of diverse helices (termed “round 2 pools”) with broad compatibility for each of the 64 different binding sites. To ensure these were enriched for functional helices and not background, a simple cutoff was devised to omit unsuccessful selections. Based on the data filtering metrics described, single-finger pools were omitted if less than 20% of the reads passed these filters as those selections would have added a disproportionate amount of non-functional ZFs to the template pools. This set of 64, round 2 pools were used as a PCR template to create either ‘domain 1’ or ‘domain 2’ amplicons using ExpandTM High Fidelity PCR system (Roche) and 15 cycles of PCR to reduce bias. ‘domain 1’ and ‘domain 2’ reactions were gel-purified from a 2% agarose gel, quantified by nanodrop, and stored at −20 C. In order to create a 2-finger library insert, overlapping PCR was performed to stitch appropriate ‘domain 1’ and ‘domain 2’ pools together. Purified single-finger amplicons were combined equimolar as the template for overlap PCR with Phusion® High Fidelity DNA Polymerase (NEB) (25 cycles), PCR-purified, digested with KpnI and NotI, gel-purified, and quantified by Nanodrop (ThermoFisher Scientific). The digested 2-finger library inserts were ligated into the 2-finger library vector (see FIG. 2D). Ligations were performed overnight at 16 C using 300 ng of digested backbone and a 5:1 molar excess insert: backbone. Ligations were ethanol precipitated and resuspended in 5 μL EB (Qiagen). 100 ng of the ligation was electroporated into USO-ω cells, recovered in SOC for 1 hr, titered on 2xYT agar plates containing 2% glucose and 100 μg/mL carbenicillin, and stored at 4 C overnight. Based on cell counts the following day, 5×10⁶ cells were plated on 15 cm rich media agar plates (2xYT, 2% glucose, 100 μg/mL carbenicillin), grown at 30 C for 12-14 hours, harvested by scraping, and finally miniprepped to obtain final round 2 libraries.

### Zinc Finger Selections

Primary ZF Libraries: Libraries were built in a vector that will express the ZFs as a fusion to the omega subunit of the bacterial polymerase using a strong promoter. In the B1H system omega is simply acting as an activation domain. The binding site reporter vectors were built by placing the binding site of interest 10 bp upstream of the −35 box of the promoter that drives HIS3 and GFP expression in the previously described GHUC vector. For example, for the library 2 TAC selection, the binding site 5′ TAC-ACA-AAG 3′ was built into the GHUC vector 10 bp upstream of the promoter where the library domain will bind TAC and domains 1 and 0 of library 2 will bind ACA and AAG, respectively (FIG. 1C). For each selection, the ΔrpoZ selection strain was transformed with the ZF library and the appropriate reporter plasmid by electroporation. The cells were expanded in 10 ml SOC for 1 h at 37 C with rotation, recovered and resuspended in minimal media supplemented with histidine and grown with rotation for an additional hour at 37 C. Finally, cells were washed in minimal media that lacks histidine, recovered in 1 ml of this media, and 20 μl's plated in serial dilution on rich plates containing Kanamycin and Carbenicillin to quantify double transformants. This plate was grown at 37 C overnight while the remaining 980 μl of transformed cells was stored at 4 C. Once grown, the serial dilutions were counted and a volume containing a minimum of 5×10⁸ cells were taken from the transformants stored at 4 C and plated on selective media. These plates contained 2 mM 3-AT, a competitive inhibitor of HIS3, that helps to removed background activity from the screen. Cells were grown on the selection plates for 36-48 h at 37 C. Colonies were counted, cells were pooled, and DNA harvested. This DNA was used as the template for Illumina sequencing. All selections resulted in hundreds to thousands of surviving colonies.

Compatible 2-finger modules selections: In order to identify compatible 2-finger modules from round 2 libraries, a matching set of vectors was first built containing the intended DNA target and then leveraged the omega-dependent activation of the HIS3 reporter in the bacteria 1-hybrid system. Round 2 libraries were co-transformed with the matching reporter vector in USO-ω cells and recovered and titered as described. Based on cell counts the next day, 1×10⁶ cells were added in triplicate to a 96-well deep-well plate containing a sterile bead for efficient agitation. Selections were performed in 1 mL NM +Ura/−His supplemented with 100 μg/mL carbenicillin, 50 μg/mL Kanamycin, 1 μM IPTG, and 5 mM 3AT. These were grown at 37 C in a plate shaker for 18, 24, or 40 hours and harvested upon reaching visible turbidity (typically OD>0.6). Triplicates were pooled, miniprepped, and deep sequenced on an Illumina NextSeq 500. Helices were rank-ordered by sequencing reads, and 2-finger modules within the top 5 highest counts were chosen for follow-up assembly and testing in the EGFP nuclease assay.

### U20S GFP Disruption Assay

Zinc finger nuclease (ZFN) activity was assessed by measuring disruption of an integrated, constitutively-expressed eGFP reporter in a clonal U2OS cell line previously described(39). Cells were cultured in DMEM supplemented with 10% FBS, 2 mM GlutaMAX™ (Life Technologies), 1% penicillin/streptomycin, 1% MEM non-essential amino acids (Life Technologies), 2 mM sodium pyruvate, and 400 μg/mL G418. 1 μg of each ZFN monomer plasmid DNA and 200 ng ptdTomato-N1 plasmid DNA were transfected in duplicate into 5×10⁵ cells using a Lonza Nucleofector™ 2b Device (Kit V, Program X-001). In each assay 2 μg of the parental empty vector (a modified derivative of the JDS71 vector from addgene) and 200 ng ptdTomato-N1 was used as a negative control, and 2 μg of a dual spCas9-guide expressing vector (modified addgene plasmid #41815) and 200 ng ptdTomato-N1 was used as a positive control in each experiment. Cells were grown in 6-well dishes for 3 days post-transfection, harvested and kept on ice, and analyzed for expression of eGFP and tdTomato on a Sony SH800 cell sorter. In order to restrict analysis to only cells that likely received both ZFN monomer plasmids, populations were first gated on the top 15-25% tdTomato+ cells, and then analyzed for loss of eGFP expression.

### Next Generation Sequencing and Prep

Primary libraries: Following selection from >5×10⁸ library variants. surviving colonies were pooled, miniprepped, and DNA barcoded for sequencing on an Illumina NextSeq® 500. Typically these were performed as a set of 64 3 bp binding sites for a given ‘overlap’ library as follows. 2 μL of pooled plasmid DNA was used as a template for barcoding in a 25 μL reaction with Taq Polymerase (NEB) with the following cycling parameters: 95 C for 5 min, 20 cycles of [95 C:20 s, 52 C:30 s, 68 C:30 s], 68 C for 10 min, and held at 4 C. 5 μL each reaction was visualized on a 1% agarose gel to confirm apparent equal amplification. All 64 reactions were pooled in equal volumes. These were run out on a 1% agarose gel, gel purified, and submitted to the NYU Genome Technology Center for sequencing on a NextSeq® 500.

2-finger libraries: Following selection of ˜3×10⁶ 2F library variants, plasmid DNA was extracted from surviving cells and barcoded for deep sequencing on an Illumina NextSeq® 500 as follows. 2 μL pooled plasmid DNA was used as a template for barcoding in a 25 μL reaction with GoTaq® Green 2× Mastermix (Promega) with the following cycling conditions: 95 C for 5 min, 15 cycles of [95 C:30 s, 68 C:30 s, 72 C:60 s], 72 C for 5 min, and held at 4 C. 10 μL each reaction was visualized on a 1% agarose gel to confirm equal amplification, all reactions were pooled in equal volumes. These were gel-purified from a 1% agarose gel, and submitted to the NYU Genome Technology Center for sequencing on an Illumina NextSeq® 500.

### Sequence Recovery and Filtering

All paired end Illumina reads are demultiplexed and trimmed into 21-mers with in-house Unix scripts based on EMBOSS 6.6.0. Trimmed DNA sequences are translated, and amino acid sequences are considered if they have at least two read counts and are coded by at least two different DNAs. The invariant Leucine at the helix position +4 is excluded.

### Clustering and Filtering Selections

For each selection, helix sequences were clustered using the MUSI software(34). Each sequence was assigned to the cluster associated with the PWM for which it was assigned the highest responsibility. For each cluster generated, the Shannon entropy value was calculated for each helix residue based on the PWM for that cluster. If a selection lacked a cluster with at least one position with an entropy of two or less, that selection was filtered out for downstream analysis.

### Computing Similarity between Selections by Hamming Distance

To compare the helices from two selections, A and B, pairwise normalized Hamming distances were computed between the two sets of filtered sequences based on the number of identical amino acids. The minimum normalized Hamming distance was then computed from each helix in selection A to each helix in selection B as well as from each helix in selection B to each helix in selection A. The overall distance between the two selections was computed as the mean of these distances.

### Molecular Dynamic Simulations

Similar to previous studies (47, 48), the PDB file 1AAY(49) was used as a template, the DNA was elongated by 2 bp at each end using X3DNA to avoid the melting end effect so that the binding of zinc fingers is not affected. The DNA and protein sequences were mutated using Chimera (www.cgl.ucsf.edu/chimera/) for each library and test case, the protonated states were determined by WHATIF (swift.cmbi.umcn.nl/whatif/) The prepared structures were then solvated into a TIP3P water box with 15-Å buffer of water extending from the protein/DNA complex in each direction, sodium ions were added to ensure the overall charge neutrality. The FF99 Barcelona forcefield was used for protein/DNA complex and zinc amber forcefield for zinc ions. The particle mesh Ewald method was used for electrostatics calculations. The SHAKE algorithm was used to constrain the hydrogen-containing bond lengths, which allowed a 2-fs time step for MD simulation. The non-bonded cut-off was set to 12.0 Å. The systems were energy minimized using a combination of steepest descent and conjugate gradient methods. Then the systems were thermalized and equilibrated for 3 ns using a multistage protocol. The first step was a 1.5 ns gradual heating from 100K to 300 K. followed by 1.5 ns of density equilibration, both at 1-fs step length. Berendsen thermostat and barostat were used for both temperature and pressure regulation for another 6-ns equilibration at 2-fs step length with gradually reduced positional constraints at 300K. The systems were built with tleap and the simulations were conducted with GPU accelerated Amber18(50). For each system, three 500-ns trajectories were simulated. The hydrogen bond analysis was performed using BioPython. Any contacts below 3.5 Å between the atoms O6 and N7 in a Guanine and the atoms NH1 and NH2 in an Arginine or ND2 and OD1 for an Asparagine were considered as hydrogen bonds. Bifurcated hydrogen bonds between a guanine and an arginine are identified when two pairs 06-NH1/2 and N7-NH1/2 are found, allowing the tautomeric bifurcated hydrogen bond.

### Calculating Entropy of Binding for Core Helices across Libraries

To quantify the promiscuity of helices that target each nucleotide three-mer, the Shannon entropy was computed. For each nucleotide three-mer, a position frequency matrix of nucleotide sequences targeted by every set of core residues (−1, 2, 3, 6) was computed. The entropy was calculated in a position wise fashion and then summed to get an overall metric for specificity.

### Neural Network Architecture

A hierarchical neural network architecture was developed that mimics the B1H experimental setup and captures the modularity of zinc finger proteins. This architecture is composed of three modules (FIG. 3). The first two modules are trained to generate helices that bind to a particular nucleotide four-mer which includes the target three-mer and the overlap base. The residue embeddings from these modules are concatenated and used as input to a third module that is designed to learn compatibility between the helices in a pair (FIG. 3). The first module generates residue embeddings for the first helix in a pair based on the last four bases in a target seven-mer and the second module generates residue embeddings for the second helix based on the first four bases in a target seven-mer (FIG. 3). The full model is trained to predict all the core residues in two helices given a nucleotide seven-mer.

The architecture of the first two modules is largely based on the Transformer model (56). An encoder generates a high-dimensional representation for each base in a nucleotide four-mer. A decoder then generates predictions for each core residue in a zinc finger helix using self-attention layers and attention layers that relate the nucleotide bases to the helix residues. While the decoder in a conventional Transformer strictly generates sequences from left to right (56), the decoders in this model use bi-directional information. A portion of the residues in a helix are masked and the decoder outputs amino acid predictions at these positions. The third module consists of repeating self-attention layers and feed forward layers that allow the model to update residue embeddings based on inter-helix compatibility (FIG. 3).

Variants of the first module with different numbers of attention heads and embedding dimensions were trained and evaluated on the initial task of predicting residues in a single helix as set out in Table 2.

TABLE 2
Accuracy of variants of the first module

| | | | | | Number | Accuracy | Accuracy | Accuracy | Accuracy |
|---|---|---|---|---|---|---|---|---|---|
| | | | | Number | of | with 3 | with 4 | with 5 | with 6 |
| | | | | of | attention | masked | masked | masked | masked |
| d_{model} | dᵥ | dₖ | dᵢ | layers | heads | residues | residues | residues | residues |
| 64 | 64 | 64 | 64 | 3 | 2 | 39.00 | 38.87 | 36.43 | 34.76 |
| 128 | 128 | 128 | 128 | 3 | 2 | 40.58 | 39.13 | 37.36 | 35.51 |
| 128 | 128 | 128 | 128 | 3 | 4 | 41.63 | 39.99 | 38.03 | 35.00 |
| 128 | 256 | 256 | 128 | 3 | 4 | 41.75 | 40.20 | 38.45 | 35.59 |
| 256 | 256 | 256 | 256 | 3 | 4 | 41.17 | 39.68 | 37.74 | 35.62 |
| 512 | 512 | 512 | 512 | 6 | 4 | 40.76 | 38.79 | 36.24 | 33.56 |

In the final model, all attention layers were repeated three times and each attention layer had four heads. The model embedding dimension (d_{model}) was set to 128. The value and key embedding dimensions for computing scaled dot-product attention (dᵥ and dₖ) were both set to 256. The hidden dimension in the feed-forward layers was set to 128. For regularization, dropout layers were included after every feed forward and attention layer with a dropout percentage of 0.3.

### Training Datasets

The models were trained and evaluated on data derived from B1H selections. B1H screening data was filtered using a previously described approach, where helices were evaluated based on the diversity of encoding nucleotide sequences found in the screen (57-59). The Shannon entropy for each helix (or helix pair) was calculated based on the number of reads associated with each possible encoding nucleotide sequence. Helices were filtered based on previously defined thresholds (58). Specifically, helices with less than ten reads or a Shannon entropy of less than 0.07 were removed.

Modules one and two were pre-trained using data from single-helix B1H selections that were performed against nucleotide four-mers. The data included selections performed with 11 libraries against 192 different nucleotide four-mers. In total, the dataset included 2,071,764 data points. For initial training and hyperparameter tuning, the data points were split into train, test, and validation datasets at proportions of 80%, 10%, and 10% respectively by four-mer sequence. For pre-training, the data was instead split by helix sequence.

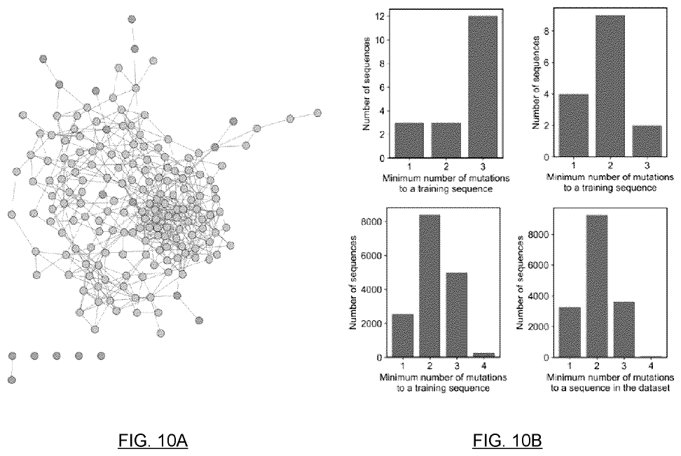

The full model was trained using data from helix-pair B1H selections that were performed against nucleotide seven-mers. An initial dataset of selections against 189 seven-mers was split into training and validation datasets at proportions of 90% and 10%. This dataset contains a total of 327,792 data points. To ensure that the validation set was sufficiently different from the training dataset, a graph was generated where nucleotide seven-mers were represented as nodes and edges connected seven-mers within two base substitutions from each other. While most of the nodes formed a single connected component, there were separate components that were included in the validation dataset (FIG. 10A). Nodes with the lowest degree in the graph, and their neighbors, were then added to the validation dataset. Most of the sequences in the validation dataset were consequently at least three mutations away from any sequence in the training dataset (FIG. 10B). A separate set of 15 selections filtered to ensure at least 100 unique helix pairs were used as an independent test set for model evaluation.

### Model Training

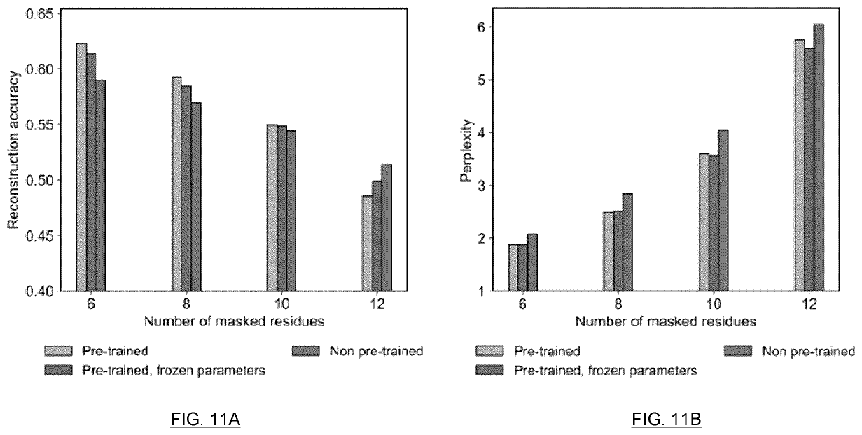

In both training steps, a nucleotide target and a sequence of partially masked core residues from either a single zinc finger or a helix pair were provided to the model. 50% of the core residues were masked and the cross-entropy loss was evaluated based on the output probabilities. Training was done using an Adam optimizer with a learning rate of 1e−4, and a minibatch size of 128 was used. Early stopping was done based on the validation loss. Pre-training modules one and two took at most 1.3 million iterations. Training the full model was at most 3.4 million iterations. When training the full model, the parameters for modules one and two were either randomly initialized, transferred from the pre-training step, or transferred and from the pre-training step and frozen (FIG. 11).

### De Novo Design of Zinc Finger Helix Pairs

When predicting zinc finger residues. the model makes use of context provided by known residues. Helix sequences are generated incrementally where the network is run once for each missing residue. At each iteration, a single residue is added to increase the sequence context. For a pair of helices, there are about 4.1×10¹⁵ possible sequences and about 4.8×10⁸ orders in which each sequence can be generated. Enumerating all possibilities to find the sequence with the highest likelihood is thus computationally intractable.

To generate sequences, the A* search algorithm was adapted (60-61). This approach involves iteratively filling in masked residues while maintaining a priority queue of partially masked sequences. At every iteration, the top partially masked sequence is taken from the priority queue and passed through the network. All possible labels for every masked residue are evaluated. Any label with a probability above 0.05 is accepted and the label is added to a copy of the input sequence before it is pushed onto the priority queue. This is repeated until a set amount of sequences are completely generated. The following equation was used to assign a priority to each partially masked sequence:

$${p}_{j} = {\sum }_{i = 1}^{j} log ( {p}_{i} ) + {\sum }_{j}^{12} log ( {p}^{*} ) \qquad ( Equation 1 )$$

This heuristic approximates the maximum expected probability of a sequence that would be attained by predicting the remaining residues. pᵢ denotes the probability assigned to the prediction made at iteration i and j denotes the number of predicted residues. p* denotes the expected maximum probability that would be assigned by the network to later predictions. This parameter can be tuned to move the search closer to a greedy search or a breadth first search. This parameter was set to 0.1 whenever A* was performed in this work.

An alternative biased sampling approach was also implemented using temperature adjusted distributions (62). This approach generally resulted in higher likelihood sequences (FIG. 12). At every iteration, the probability of predicting an amino acid i at position j is the following:

$${p ( {x}_{i , j} \vert n , {x}_{( k , m ) ∈ S} )}^{( T )} = \frac{{p ( {x}_{i , j} \vert n , {x}_{( k , m ) ∈ S} )}^{\frac{1}{T}}}{{\sum }_{a = 1}^{20} {\sum }_{b = 1}^{12} {p ( {x}_{a , b} \vert n , {x}_{( k , m ) ∈ S} )}^{\frac{1}{T}}} \qquad ( Equation 2 )$$

n denotes the input nucleotide sequence and S denotes the set of pairs of amino acids and positions that have already been predicted. T is an adjustable parameter that controls the bias of the distribution. This parameter was set to 0.6 when this method was used. 10⁵ ZF pairs were sampled and the maximum likelihood pair when performing de novo design.

### Comparison to ZFPred

To generate distributions over helix sequences using ZFPred³, 10⁶ helix sequences were randomly sampled. The binding specificities of these helices were predicted using ZFPred. Sequence distributions for a particular nucleotide sequence were then generated by normalizing the predicted scores of the sampled helices for that nucleotide sequence. Predictions for 3-mers were concatenated to generate predictions for 6-mer sequences.

### Live Cell Imaging of ZF-GFP Fusion

Zinc fingers were designed to bind the sequence 5′-CGCCCAGCTGGGGGCGGGGGA-3′, a sequence that is repeated 111 times at the Brf1 locus on chromosome 14 (hg38 chr14: 105229626-105240946). The coding sequence for the designed zinc finger array was ordered from IDT (gBlock) A SV40 NLS was added to the C-termini by PCR. Next, GFP was added as an N-terminal fusion to the zinc fingers using the NT-GFP Fusion TOPO TA Expression Kit (Invitrogen). Successful cloning into the expression vector was confirmed by Sanger sequencing.

The GFP-ZF fusion expression vector was transfected into 293T cells and grown on 0.01% Poly-L-Lysine coated 35 mm MatTek dishes using X-treme-GENE 9 DNA transfection reagent (Sigma Aldrich). Transfected cells were Hoechst stained the next day and then imaged. A titration experiment was conducted to explore optimal plasmid concentration. Clear foci were visible at a range of concentrations, but 333 ng of plasmid yielded the optimal balance of transfection efficiency and signal to noise ratio.

### Cell Culture and RT-qPCR Analysis of Repressors and Activators

HEK293T cells were transfected with ZF-repressors, ZF-activators, or SpCas9-repressors targeting various endogenous loci and target transcript levels were measured by RT-qPCR as follows. 2 μg of the parental (pKJ-Kan) plasmid DNA or 2 μg of pMMBC_SpCas9 containing a non-targeting guide were used as negative controls for ZF and SpCas9 transfections, respectively. Cells were cultured in DMEM supplemented with 10% FBS, 2 mM GlutaMAX™ (Life Technologies), 1% penicillin/streptomycin, 1% MEM non-essential amino acids (Life Technologies) and 2 mM sodium pyruvate. 18-24 hours prior to transfection, cells were passaged and 7.5e5 cells were added to 2.5 mL media in a 6-well dish. Cells were transfected with 2 μg of plasmid DNA using a 4:1 ratio of DNA: TransIT®-LT1 transfection reagent (Mirus) according to manufacturer's instructions. Media was changed 2 days post-transfection, and cells were harvested for RT-qPCR 3 days post-transfection. Cells were washed once with sterile PBS, 350 μL Buffer RLT Plus (Qiagen) containing 1% β-mercaptoethanol was added, and samples were either stored at −80 C or processed immediately using a RNeasy Plus Mini Kit (Qiagen) according to manufacturer's instructions. Pure RNA was quantified using a NanoDrop™ 2000c (Thermo Scientific™) and stored at −80 C.

1 μg of pure RNA was reverse transcribed using the SuperScript™ IV First-Strand Synthesis System (Invitrogen™) according to manufacturer's instructions except half the recommended reverse transcriptase was used. Random hexamers were used as primers, and cDNA was stored at −20 C or processed immediately. qPCR reactions were set up in technical duplicate or triplicate using the equivalent of 25 ng or 50 ng reverse-transcribed RNA per reaction and the KAPA SYBR FAST qPCR Master Mix (2×) (Roche).

RT-qPCR was performed on a LightCycler® 480 Instrument II (Roche) using the cycling program recommended for KAPA SYBR FAST reagent on the LightCycler® 480 (annealing temperature was 60 C). Ct values were calculated using the on-board “Absolute Quantification/2^{nd} Derivative Max” analysis option. Input was first normalized using the housekeeping gene RPS18, and fold-change in expression for a given gene of interest was calculated relative to the appropriate negative control.

### RNA-seq Analysis

RNA-Seq library preps were constructed using the Illumina TruSeq® Stranded mRNA Library Prep kit (Cat #20020595) using 500-1000 ng of total RNA as input, amplified by 10-12 cycles of PCR, and sequenced paired-end 50 cycles on Illumina sequencers with 2% PhiX spike-in. 25-30 million reads were obtained for each sample. Paired-end reads were aligned to hg38 using STAR aligner (63). Read counts were computed using FeatureCounts and differential expression analysis was subsequently performed using DESeq2 (64).

### Example 2: De Novo Design of Extended Zinc Finger Arrays

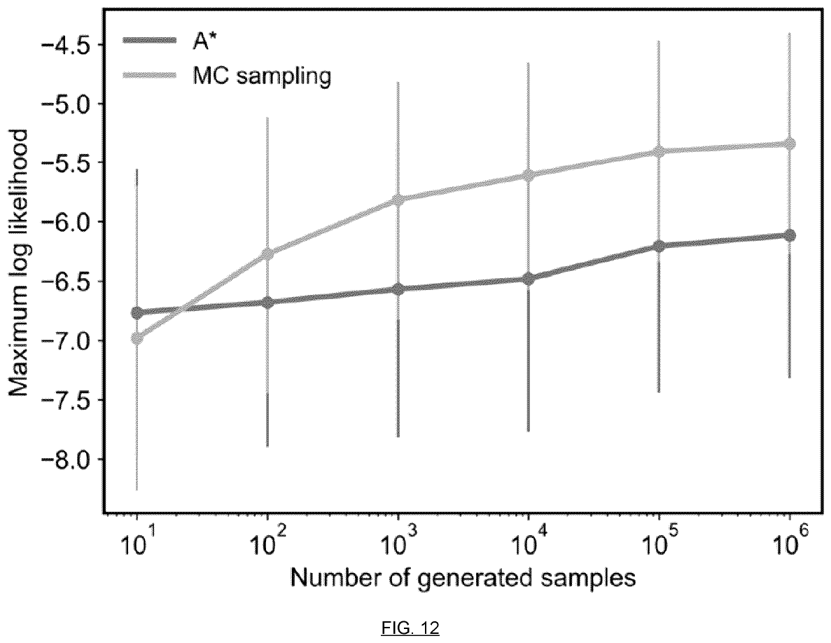

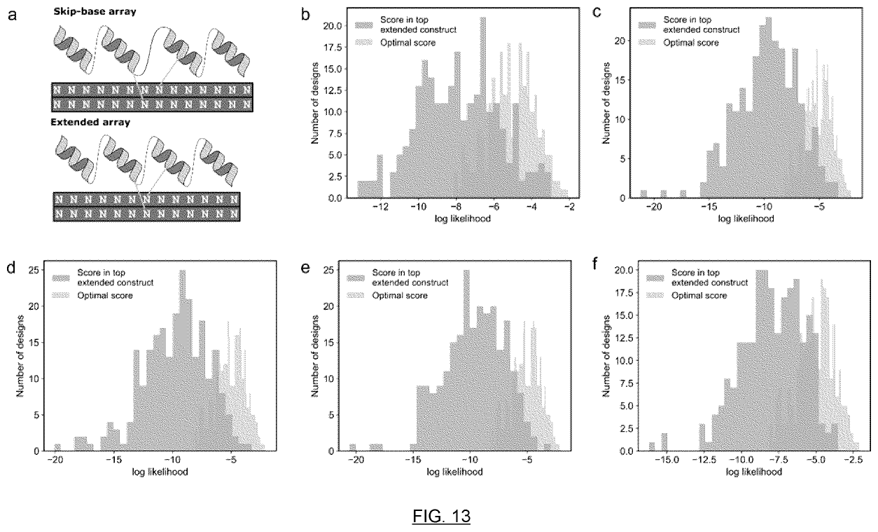

Natural ZFs occur in arrays that contain more than two fingers. All the helix pairs in these arrays must be mutually compatible for the ZFs to bind their target DNA molecules. It was hypothesized that the use of a flexible linker between helix pairs negatively impacts ZF binding due to an additional entropic cost and potential off-target effects due to the independent binding of individual helix pairs. Based on this, alternate versions of the ZF sampling procedures were developed to generate so called “extended arrays” that lack the flexible linkers included in “skip-base arrays” (FIG. 13A). With these augmented search procedures, n helices are designed to bind a target sequence of length 3n+1. An array of n helices contains n−1 pairs. Instead of running the model once per iteration when performing A* or MC sampling, the model is run n−1 times, one for each helix pair. To ensure that overlapping helix pairs are mutually compatible, labels at residues that occur in two helix pairs are assigned the minimum probabilities predicted in the two runs of the model. This represents the most stringent aggregation of probabilities.

Given that additional constraints are placed during sampling, the predicted likelihoods of the generated helix pairs in the extended format are lower. Six-finger extended array constructs generated for different 19-mer targets show a consistent drop-off in the likelihood across all five helix pairs (FIGS. 13B-F). To test whether the reduced flexibility compensated for the decrease in the predicted binding of individual helix pairs, extended array ZFs were designed for different experimental assays. ZF nucleases localized with four-finger extended arrays were found to effectively cut GFP (FIG. 14A). ZF nucleases with four-finger and six-finger arrays were also designed to target the CCR5 endogenous locus. The T7 endonuclease assay was used to quantify the cutting of these nucleases and it was found that they showed comparable cutting to previously designed ZF nucleases(55) (FIG. 14B). Additionally, extended arrays were designed to target the repressor ZIM3 near the TSS of different genes. Western blots against alpha-synuclein (SNCA) show that most of the designed ZFs successfully repress SNCA (FIG. 15A). RT-qPCR furthermore demonstrated that designed ZFs could be used to repress mouse SNCA (FIG. 15B). Finally, designed ZFs were also used to repress TDP43 (FIG. 15C).

TABLE 3
Designed extended array ZF sequences

| Gene | | Construct | | | | | | |
|---|---|---|---|---|---|---|---|---|
| Target | Target | Type | Helix 1 | Helix 2 | Helix 3 | Helix 4 | Helix 5 | Helix 6 |
| GFP | 1 | Nuclease | QKSNLKR | LKHHLTR | DRSTLRQ | DPSNLRR | — | — |
| | | left | | | | | | |
| | | monomer | | | | | | |
| GFP | 1 | Nuclease | RKFNLLR | DPSALNR | RKDVLLG | DPSALNR | — | — |
| | | right | | | | | | |
| | | monomer | | | | | | |
| GFP | 2 | Nuclease | QSGTLYN | RKAHLRD | RKDRLRH | WKCDLVR | — | — |
| | | left | | | | | | |
| | | monomer | | | | | | |
| GFP | 2 | Nuclease | LKQTLQS | RRGDLNR | RKFTLRQ | RRGTLRR | — | — |
| | | right | | | | | | |
| | | monomer | | | | | | |
| GFP | 13 | Nuclease | RKWLNR | TRRYLHS | RDPSNLNR | RRYGLRR | — | — |
| | | left | | | | | | |
| | | monomer | | | | | | |
| GFP | 13 | Nuclease | HPSTLRN | RKYSLLR | FPYLLRN | DRSTLRR | — | — |
| | | right | | | | | | |
| | | monomer | | | | | | |
| GFP | 15 | Nuclease | DPSNLLRR | KWTLKM | RKFTLQC | QSRYLRR | — | — |
| | | left | | | | | | |
| | | monomer | | | | | | |
| GFP | 15 | Nuclease | QKSNLLR | LKWNLKS | RRGDLNR | RRDRLRH | — | — |
| | | right | | | | | | |
| | | monomer | | | | | | |
| CCR5 | 1A | Nuclease | HPSTLRN | RKSTLQA | RKYHLRQ | RKSTLQS | QLRYLSR | TSSGLCH |
| | | left | | | | | | |
| | | monomer | | | | | | |
| CCR5 | 1A | Nuclease | SKQNLQN | RTSNLRR | QKSNLLR | LKQTLTR | RKWLTKM | QSGNLRS |
| | | right | | | | | | |
| | | monomer | | | | | | |
| CCR5 | 1B | Nuclease | HPSTLRN | RKSTLQA | RKYHLRQ | RKSTLQS | HKRNLSA | TSSGLCH |
| | | left | | | | | | |
| | | monomer | | | | | | |
| CCR5 | 1B | Nuclease | SKQNLQN | RTSNLRR | QKSNLLR | LKQTLSR | RKWTLKM | QSGNLRS |
| | | right | | | | | | |
| | | monomer | | | | | | |
| CCR5 | 2A | Nuclease | RKYHLQQ | RKSTLQS | RKYHLQQ | TSSGLCH | — | — |
| | | left | | | | | | |
| | | monomer | | | | | | |
| CCR5 | 2A | Nuclease | QKSNLKR | LKQTLTR | RKWTLKM | QSGNLRS | — | — |
| | | right | | | | | | |
| | | monomer | | | | | | |
| CCR5 | 2B | Nuclease | RKYHLQQ | RKSTLQN | RKYHLQQ | TSSGLCH | — | — |
| | | left | | | | | | |
| | | monomer | | | | | | |
| CCR5 | 2B | Nuclease | QKSNLKR | LKQTLSR | RKWTLKM | QSGNLRS | — | — |
| | | right | | | | | | |
| | | monomer | | | | | | |
| SNCA | 1 | Repressor | RKAHLRD | RKSTLRS | RRGDLLR | DRSTLRR | FPYLLRR | RRSTLRS |
| SNCA | 2 | Repressor | RRGDLNR | FPYLLRR | YPYLLRA | RRQTLRD | RKSTLRD | TSQSLSY |
| SNCA | 3 | Repressor | YPYLLRR | YPYLLRN | RKQTLQD | RKSTLRD | SRQSLNY | QYSSLYK |
| SNCA | 4 | Repressor | LKHHLLS | QKAHLLR | YPYLLRN | QKAHLSA | RRYDLRM | RRTTLRD |
| SNCA | 5 | Repressor | QKVHLLR | YPYLLRN | QKAHLTS | RKYDLRM | RRTTLRD | QKVHLRS |
| SNCA | 6 | Repressor | QKSNLKT | RKFTLQC | RRGDLNR | QKVDLNR | YKFVLRS | WRSSLVA |
| SNCA | 7 | Repressor | YPYLLRH | QKAHLTS | RRYDLRM | RRTTLRD | QKVHLLS | DPSNLRR |
| SNCA | 8 | Repressor | RKSTLRS | RKGTLLR | DRSTLRR | FPYLLRR | RKDTLLS | RRDRLRH |
| SNCA | 9 | Repressor | QKAHLLA | RKYDLRM | RRTTLRD | QKVHLLS | DPSNLRR | FPYLLRR |
| SNCA | 10 | Repressor | RKWNLLT | VKRRLVN | QKAHLLR | LKQTLQR | QKVHLVT | TSSHLCH |
| SNCA | 11 | Repressor | RKWDLRQ | RRSTLRD | QKAHLLA | DPSNLNR | FPYLLRR | RRSTLRD |
| SNCA | 12 | Repressor | RADRLRQ | RTYNLLR | DRTTLRR | RRGDLNR | QKVHLLS | RRTHLRD |
| SNCA | 13 | Repressor | RKDTLRN | DRSTLRR | FPYLLRR | RKDTLRD | RADRLRH | RTTNLRR |
| SNCA | 14 | Repressor | DPSNLLR | YPYLLRS | RADRLRH | RTYNLSR | DRSTLRR | RRGDLRR |
| SNCA | 15 | Repressor | RKFNLLR | DRTTLRR | RKGDLNR | QKVHLQS | RKYHLSR | RRYSLSA |
| SNCA | 16 | Repressor | FPYLLRN | RKATLRD | QKAHLTA | RKWNLLR | FPYLLRR | RRYSLRC |
| SNCA | 17 | Repressor | RKFSLRN | SSSNLLR | LKHHLTS | QKAHLSR | FPYLLRN | RRDRLRS |
| SNCA | 18 | Repressor | RKSNLLR | LKHHLTS | QKAHLTR | YPYLLRN | QKAHLTA | RRSTLRQ |
| SNCA | 19 | Repressor | DRSTLRN | FPYLLRR | RKDTLRS | QKAHLLA | RKSNLNR | ERSKLRR |
| SNCA | 20 | Repressor | YPYLLRH | RRDRLRA | RLYNLSR | DRSTLRR | RRGDLHR | QSTHLRA |
| Mouse | 36 | Repressor | RRGDLNR | RKFTLLC | RRDRLRN | WKVDLKR | DRSTLRR | DRSTLRR |
| SNCA | | | | | | | | |
| Mouse | 38 | Repressor | RKFTLQS | RRDRLRH | WKVDLSR | DRSTLRR | DRSTLRQ | RRDRLRR |
| SNCA | | | | | | | | |
| Mouse | 54 | Repressor | RRDRLLK | DPSTLRR | FPYLLRN | DRSTLRQ | RRYDLRM | TSSNLSK |
| SNCA | | | | | | | | |
| Mouse | 73 | Repressor | DPSTLRR | HKHHLTG | WKIDLLR | RKWVLQC | RKDRLRH | WKIDLVR |
| SNCA | | | | | | | | |
| TDP43 | 1 | Repressor | DRTTLRR | RKAHLRE | QKAHLKS | RKWNLKM | RKWTLKM | RRSTLRS |
| TDP43 | 2 | Repressor | RKAHLRD | QKSHLTA | RKWNLRM | RKWTLKM | RRSTLRD | QSGTLHR |
| TDP43 | 3 | Repressor | QKAHLKS | RKWNLLM | RKWTLLM | RKSTLRD | QSGTLYN | RRAHLRD |
| TDP43 | 4 | Repressor | RKDRLLK | FPYLLRR | RKWTLKM | DRSTLRQ | QSSHLRR | DRSNLRR |
| TDP43 | 6 | Repressor | TSSNLAH | QKVHLLS | DPSNLNR | QSSHLTR | RKWNLKQ | RRDRLLS |
| TDP43 | 7 | Repressor | RRSTLRD | QKSNLRS | DRSTLRR | RKAHLLS | QKSHLKS | RKWNLRM |
| TDP43 | 8 | Repressor | RKWNLLM | RRTTLRD | QKVNLLS | DRSTLRR | RKAHLRE | RADRLRH |
| TDP43 | 9 | Repressor | QKVHLQS | DPSNLNR | QSSHLTR | RKWNLKM | QKAHLTG | HYKSLWR |
| TDP43 | 10 | Repressor | RKWTLKQ | RKSTLRD | QSGTLYN | RRYHLSR | QKVTLLR | QRRYLTT |
| TDP43 | 11 | Repressor | QSGTLYN | RRYHLSR | QKSTLVR | RKSTLRD | TKQYLSR | QKAHLVR |
| TDP43 | 12 | Repressor | QKVNLLS | DRSTLRR | RKAHLRE | QKAHLTA | RKWNLRM | RKWTLSM |

All publications, biological sequences or sequence identifiers, patents and patent applications are herein incorporated by reference in their entirety to the same extent as if each individual publication, biological sequence, patent or patent application was specifically and individually indicated to be incorporated by reference in its entirety.

### REFERENCES

1. N. Matharu et al., CRISPR-mediated activation of a promoter or enhancer rescues obesity caused by haploinsufficiency. Science 363, (2019).
2. A. A. Dominguez, W. A. Lim, L. S. Qi, Beyond editing: repurposing CRISPR-Cas9 for precision genome regulation and interrogation. Nat Rev Mol Cell Biol 17, 5-15 (2016).
3. B. Chen, R. B. Altman, Opportunities for developing therapies for rare genetic diseases: focus on gain-of-function and allostery. Orphanet J Rare Dis 12, 61 (2017).
4. L. A. Gilbert et al., Genome-Scale CRISPR-Mediated Control of Gene Repression and Activation. Cell 159, 647-661 (2014).
5. P. Perez-Pinera et al., RNA-guided gene activation by CRISPR-Cas9-based transcription factors. Nat Methods 10, 973-976 (2013).
6. P. I. Thakore, C. A. Gersbach, Design, Assembly, and Characterization of TALE-Based Transcriptional Activators and Repressors. Methods Mol Biol 1338, 71-88 (2016).
7. P. I. Thakore et al., Highly specific epigenome editing by CRISPR-Cas9 repressors for silencing of distal regulatory elements. Nat Methods 12, 1143-1149 (2015).
8. A. Amabile et al., Inheritable Silencing of Endogenous Genes by Hit-and-Run Targeted Epigenetic Editing. Cell 167, 219-232 e214 (2016).
9. J. K. Nunez et al., Genome-wide programmable transcriptional memory by CRISPR-based epigenome editing. Cell 184, 2503-2519 e2517 (2021).
10. M. Jinek et al., A programmable dual-RNA-guided DNA endonuclease in adaptive bacterial immunity. Science 337, 816-821 (2012).
11. C. T. Charlesworth et al., Identification of preexisting adaptive immunity to Cas9 proteins in humans. Nat Med 25, 249-254 (2019).
12. D. L. Wagner et al., High prevalence of Streptococcus pyogenes Cas9-reactive T cells within the adult human population. Nat Med 25, 242-248 (2019).
13. C. Anders. O. Niewoehner, A. Duerst, M. Jinek, Structural basis of PAM-dependent target DNA recognition by the Cas9 endonuclease. Nature 513, 569-573 (2014).
14. H. Nishimasu et al., Crystal structure of Cas9 in complex with guide RNA and target DNA. Cell 156, 935-949 (2014).
15. I. Sadowski, J. Ma, S. Triezenberg, M. Ptashne, GAL4-VP16 is an unusually potent transcriptional activator. Nature 335, 563-564 (1988).
16. A. Chavez et al., Highly efficient Cas9-mediated transcriptional programming. Nat Methods 12, 326-328 (2015).
17. C. C. Wilkens M S, Pearl J. Schanzer E, Liao H, Van Biber B, Quietsch K, Bloom J, Federation A, Acosta R, Vong S, Otterman E, Dunn D, Wang H, Zraszhevskiy P, Nandakumar V, Bates D, Sandstrom R, Urnov FD, Funnell A, Green S, and Stamatoyannopoulos J A, Quantitative dialing of gene expression via precision targeting of KRAB repressors. BioRxiv, (2021).
18. S. A. Wolfe, L. Nekludova, C. O. Pabo, DNA recognition by Cys2His2 zinc finger proteins. Annu Rev Biophys Biomol Struct 29, 183-212 (2000).
19. A. Klug, The discovery of zinc fingers and their applications in gene regulation and genome manipulation. Annu Rev Biochem 79, 213-231 (2010).
20. S. A. Lambert et al., The Human Transcription Factors. Cell 175, 598-599 (2018).
21. M. Imbeault, P. Y. Helleboid, D. Trono, KRAB zinc-finger proteins contribute to the evolution of gene regulatory networks. Nature 543, 550-554 (2017).
22. S. V. Razin, V. V. Borunova. O. G. Maksimenko, O. L. Kantidze, Cys2His2 zinc finger protein family: classification, functions, and major members. Biochemistry (Mosc) 77, 217-226 (2012).
23. S. Sydor et al., Kruppel-like factor 6 is a transcriptional activator of autophagy in acute liver injury. Sci Rep 7. 8119 (2017).
24. H. A. Greisman, C. O. Pabo, A general strategy for selecting high-affinity zinc finger proteins for diverse DNA target sites. Science 275, 657-661 (1997).
25. M. Isalan, A. Klug, Y. Choo, A rapid, generally applicable method to engineer zinc fingers illustrated by targeting the HIV-1 promoter. Nat Biotechnol 19, 656-660 (2001).
26. D. J. Segal, B. Dreier, R. R. Beerli, C. F. Barbas, 3rd, Toward controlling gene expression at will: selection and design of zinc finger domains recognizing each of the 5′-GNN-3′ DNA target sequences. Proc Natl Acad Sci U S A 96, 2758-2763 (1999).
27. M. L. Maeder et al., Rapid “open-source” engineering of customized zinc-finger nucleases for highly efficient gene modification. Mol Cell 31, 294-301 (2008).
28. A. Gupta et al., An optimized two-finger archive for ZFN-mediated gene targeting. Nat Methods 9, 588-590 (2012).
29. Y. Choo, A. Klug, Toward a code for the interactions of zinc fingers with DNA: selection of randomized fingers displayed on phage. Proc Natl Acad Sci U S A 91, 11163-11167 (1994).
30. B. Dreier, R. R. Beerli, D. J. Segal, J. D. Flippin, C. F. Barbas, 3rd, Development of zinc finger domains for recognition of the 5′-ANN-3′ family of DNA sequences and their use in the construction of artificial transcription factors. J Biol Chem 276, 29466-29478 (2001).
31. B. Dreier et al., Development of zinc finger domains for recognition of the 5′-CNN-3′ family DNA sequences and their use in the construction of artificial transcription factors. J Biol Chem 280, 35588-35597 (2005).
32. E. J. Rebar, C. O. Pabo, Zinc finger phage: affinity selection of fingers with new DNA-binding specificities. Science 263, 671-673 (1994).
33. C. Zhu et al., Using defined finger-finger interfaces as units of assembly for constructing zinc-finger nucleases. Nucleic Acids Res 41, 2455-2465 (2013).
34. T. Kim et al., MUSI: an integrated system for identifying multiple specificity from very large peptide or nucleic acid data sets. Nucleic Acids Res 40, e47 (2012).
35. A. L. Mueller et al., The geometric influence on the Cys2His2 zinc finger domain and functional plasticity. Nucleic Acids Res 48, 6382-6402 (2020).
36. A. R. Leach, A. P. Lemon, Exploring the conformational space of protein side chains using dead-end elimination and the A* algorithm. Proteins 33, 227-239 (1998).
37. G. V. Ingraham J, Barzilay R. and Jaakkola T., in Advnaces of Neural Information Processing Systems 32. (2019).
38. E. M. Handel et al., Versatile and efficient genome editing in human cells by combining zinc-finger nucleases with adeno-associated viral vectors. Hum Gene Ther 23, 321-329 (2012).
39. D. Reyon et al., FLASH assembly of TALENs for high-throughput genome editing. Nat Biotechnol 30, 460-465 (2012).
40. B. P. Kleinstiver et al., Engineered CRISPR-Cas9 nucleases with altered PAM specificities. Nature 523, 481-485 (2015).
41. D. E. Paschon et al., Diversifying the structure of zinc finger nucleases for high-precision genome editing. Nat Commun 10, 1133 (2019).
42. M. S. Bhakta et al., Highly active zinc-finger nucleases by extended modular assembly. Genome Res 23, 530-538 (2013).
43. N. Alerasool, D. Segal, H. Lee, M. Taipale, An efficient KRAB domain for CRISPRi applications in human cells. Nat Methods 17, 1093-1096 (2020).
44. A. S. Khalil et al., A synthetic biology framework for programming eukaryotic transcription functions. Cell 150, 647-658 (2012).
45. J. C. Miller et al., Enhancing gene editing specificity by attenuating DNA cleavage kinetics. Nat Biotechnol 37. 945-952 (2019).
46. A. V. Persikov, E. F. Rowland, B. L. Oakes, M. Singh, M. B. Noyes, Deep sequencing of large library selections allows computational discovery of diverse sets of zinc fingers that bind common targets. Nucleic Acids Res 42, 1497-1508 (2014).
47. A. L. Mueller et al., The geometric influence on the Cys2His2 zinc finger domain and functional plasticity. Nucleic Acids Research 48, 6382-6402 (2020).
48. M. Garton et al., A structural approach reveals how neighbouring C2H2 zinc fingers influence DNA binding specificity. Nucleic Acids Research 43, 9147-9157 (2015).
49. M. Elrod-Erickson. M. A. Rould, L. Nekludova, C. O. Pabo, Zif268 protein&#x2013; DNA complex refined at 1.6& #xe5;: a model system for understanding zinc finger&#x2013;DNA interactions. Structure 4, 1171-1180 (1996).
50. D. A. Case et al., The Amber biomolecular simulation programs. Journal of computational chemistry 26, 1668-1688 (2005).
51. M. Elrod-Erickson, M. A. Rould, L. Nekludova, C. O. Pabo, Zif268 protein-DNA complex refined at 1.6 A: a model system for understanding zinc finger-DNA interactions. Structure 4, 1171-1180 (1996).
52. A. V. Persikov et al., A systematic survey of the Cys2His2 zinc finger DNA-binding landscape. Nucleic Acids Res 43. 1965-1984 (2015).
53. M. Isalan, A. Klug, Y. Choo, Comprehensive DNA recognition through concerted interactions from adjacent zinc fingers. Biochemistry 37, 12026-12033 (1998).
54. L. Reynolds et al., Repression of the HIV-1 5′ LTR promoter and inhibition of HIV-1 replication by using engineered zinc-finger transcription factors. Proc Natl Acad Sci U S A 100, 1615-1620 (2003).
55. Oakes, B. L. et al. Multi-reporter selection for the design of active and more specific zinc-finger nucleases for genome editing. Nat. Commun. 7, 10194 (2016).
56. Vaswani, A. et al. Attention is all you need. in Advances in Neural Information Processing Systems (2017).
57. Persikov, A. V et al. A systematic survey of the Cys2His2 zinc finger DNA-binding landscape. Nucleic Acids Res. 43, 1965-1984 (2015).
58. Mueller, A. L. et al. The geometric influence on the Cys2His2 zinc finger domain and functional plasticity. Nucleic Acids Res. 48, 6382-6402 (2020).
59. Persikov, A. V, Rowland, E. F., Oakes, B. L., Singh, M. & Noyes, M. B. Deep sequencing of large library selections allows computational discovery of diverse sets of zinc fingers that bind common targets. Nucleic Acids Res. 42, 1497-1508 (2013).
60. Leach, A. R. & Lemon, A. P. Exploring the conformational space of protein side chains using dead-end elimination and the A* algorithm. Proteins Struct. Funct. Bioinforma. 33, 227-239 (1998).
61. Strokach, A., Becerra, D., Corbi, C., Perez-Riba, A. & Kim, P. M. Designing real novel proteins using deep graph neural networks. bioRxiv 868935 (2019) doi:10.1101/868935.
62. Ingraham, J., Garg, V. K., Barzilay, R. & Jaakkola, T. Generative models for graph-based protein design. in Deep Generative Models for Highly Structured Data, DGS@ICLR 2019 Workshop (2019).
63. Dobin, A. et al. STAR: ultrafast universal RNA-seq aligner. Bioinformatics 29, 15-21 (2013).
64. Love, M. I., Huber, W. & Anders, S. Moderated estimation of fold change and dispersion for RNA-seq data with DESeq2. Genome Biol. (2014) doi:10.1186/s13059-014-0550-8.

1. A method for determining a Zinc Finger (ZF) protein sequence for binding a target nucleic acid sequence, the method comprising:
providing, in a memory, a ZF protein design model comprising a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence;
receiving as input a target nucleic acid sequence at a processor in communication with the memory, the target nucleic acid sequence comprising the first target nucleic acid subsequence and the second target nucleic acid subsequence, wherein the first target nucleic acid subsequence and the second target nucleic acid subsequence overlap;
determining a first embedding at the processor, the first embedding based on the first target nucleic acid subsequence and the first module of the protein design model;
determining a second embedding at the processor, the second embedding based on the second target nucleic acid subsequence and the second module of the protein design model; and
determining the ZF protein sequence at the processor, the ZF protein sequence based on the first embedding, the second embedding, and the third module of the protein design model.

2. The method of claim 1, wherein the first module, the second module, and the third module comprise attention-based deep learning models;
wherein the first module, the second module, and the third module comprise recurrent neural networks with long short-term memory;
wherein the first module and the second module comprise encoder models and the third module comprises a decoder model, optionally wherein the encoder models generate a high-dimensional representation for each DNA base in the target nucleic acid sequence and the decoder model generates predictions for each amino acid residue in the ZF protein sequence; and/or
wherein the first embedding and the second embedding are concatenated prior to input to the third module.

3-5. (canceled)

6. The method of claim 1, wherein the third module comprises at least one self-attention layer and at least one feed forward layer, optionally wherein the at least one self-attention layer comprises at least three self-attention layers, the at least one feed forward layer comprises at least three self-attention layers, and each self-attention layer comprises at least four heads, optionally wherein:
the concatenation of the first embedding and the second embedding comprises an embedding dimension of 128;
a value and a key embedding for computing scaled dot-product attention in the at least one self-attention layers comprises 256 dimensions; and/or
a hidden dimension in the at least one feed-forward layers comprises 128 dimensions.

7-8. (canceled)

9. The method of claim 1, wherein the ZF protein design model is executed iteratively to incrementally determine the ZF protein sequence, optionally wherein a single amino acid in the ZF protein sequence is determined per iteration of the ZF protein design model.

10. The method of claim 9, wherein:
the determining the first embedding and the second embedding at the processor further comprises receiving a first masked ZF protein subsequence and a second masked ZF protein subsequence; and
the iterative execution of the ZF protein design model comprises reducing the size of a mask of the first and second masked ZF protein subsequences; or
wherein determining the candidate protein sequence further comprises executing an iteration of a search algorithm based on the first masked ZF protein subsequence, the second masked ZF protein subsequence, and the protein design model, optionally wherein the search algorithm is the A* search algorithm, and the executing the iteration of the search algorithm further comprises:
maintaining a priority queue of one or more partially masked ZF protein sequences; and
determining a probability of a top partially masked ZF protein sequence in the priority queue by processing the top partially masked ZF protein sequence using the protein design model, optionally wherein the probability of the top partially masked ZF protein sequence is determined using the equation: pⱼ=Σᵢ₌₁^{j}log(pᵢ)+Σⱼ¹²log(p*).

11-13. (canceled)

14. The method of claim 10, wherein the probability of the top partially masked ZF protein sequence is determined using Monte Carlo sampling, optionally using the equation:
$${p ( {x}_{i , j} \vert n , {x}_{( k , m ) ∈ S} )}^{( T )} = \frac{{p ( {x}_{i , j} \vert n , {x}_{( k , m ) ∈ S} )}^{\frac{1}{T}}}{{\sum }_{a = 1}^{20} {\sum }_{b = 1}^{12} {p ( {x}_{a , b} \vert n , {x}_{( k , m ) ∈ S} )}^{\frac{1}{T}}}$$

15. The method of claim 1, wherein the first target nucleic acid subsequence and the second target nucleic acid subsequence are 4 nucleotides in length, wherein the 5′ nucleotide of the first target nucleic acid subsequence and the 3′ nucleotide of the second target nucleic acid sequence overlap;
wherein the target nucleic acid sequence is 7 nucleotides in length and the ZF protein sequence defines a ZF helix pair that binds to a target nucleic acid comprising the target nucleic acid sequence;
wherein the first ZF protein subsequence and/or second ZF protein subsequence each comprises a set of 6 amino acid residues, optionally wherein the set of 6 amino acid residues define a DNA-binding domain; and/or
wherein the ZF protein sequence is an extended array comprising n helices and the target nucleic acid sequence has a target sequence of length 3n+1, optionally wherein the ZF protein design model is run n−1 times, one for each helix pair in the extended array of n helices.

16-19. (canceled)

20. The method of claim 1, wherein the first module and second module are trained on single helix ZF specificity data, optionally wherein the single helix ZF specificity data comprises data on single ZF helix protein sequences that bind polynucleotides comprising or consisting of a target 4-mer;
wherein the third module is trained on ZF helix-pair binding data, optionally wherein the ZF helix-pair binding data comprises data on ZF helix-pair sequences that bind polynucleotides comprising or consisting of a target 7-mer; and/or
wherein the method furhter comprises synthesizing a polypeptide comprising an amino acid sequence based on the the ZF protein sequence or a nucleic acid molecule encoding said polypeptide.

21-22. (canceled)

23. A method of reprogramming a biomolecule to bind a target nucleic acid sequence, the method comprising:
providing a biomolecule, or a nucleic acid encoding a biomolecule;
modifying the biomolecule, or the nucleic acid encoding the biomolecule, to bind the target nucleic acid sequence based on one or more ZF protein sequences, wherein the one or more ZF protein sequences are determined according to the method of claim 1.

24. A system for determining a Zinc Finger (ZF) protein sequence for binding a target nucleic acid sequence, the system comprising:
a memory, the memory comprising:
a ZF protein design model comprising a first module for predicting a first ZF protein subsequence that binds with a first target nucleic acid subsequence, a second module for predicting a second ZF protein subsequence that binds with a second target nucleic acid subsequence and a third module for predicting the ZF protein sequence based on the first ZF protein subsequence and the second ZF protein subsequence;
a processor in communication with the memory, the processor configured to:
receive as input a target nucleic acid sequence, the target nucleic acid sequence comprising the first target nucleic acid subsequence and the second target nucleic acid subsequence, wherein the first target nucleic acid subsequence and the second target nucleic acid subsequence overlap;
determine a first embedding based on the first target nucleic acid subsequence and the first module of the protein design model;
determine a second embedding based on the second target nucleic acid subsequence and the second module of the protein design model;
determine the ZF protein sequence based on the first embedding, the second embedding, and the third module of the protein design model, optionally wherein the processor is configured to determine the ZF protein sequence for binding the target nucleic acid sequence according to the method of claim 1.

25. (canceled)

26. A method of generating a model for determining a Zinc Finger (ZF) protein sequence for binding a target nucleic acid sequence, the method comprising:
providing a hierarchical machine learning model comprising:
a first layer comprising a first module and a second module; and
a second layer comprising a third module,
wherein embeddings from the first layer are fed into the second layer;
training the first module based on single helix binding data, wherein the single helix binding data comprises data on single ZF helices that bind polynucleotides comprising a target 3-mer and one or more adjacent nucleotides in the 5′ position;
training the second module based on single helix binding data, wherein the single helix binding data comprises data on single ZF helices that bind polynucleotides comprising a target 3-mer and one or more adjacent nucleotides in the 3′ position;
training the hierarchical machine learning model based on helix-pair binding data, wherein the helix pair specificity data comprises data on ZF-helix pairs that bind polynucleotides comprising a target 6-mer.

27. The method of claim 26, wherein the first module, the second module, and the third module comprise attention-based deep learning models and/or wherein the first module, the second module, and the third module comprise recurrent neural networks with long short-term memory.

28. (canceled)

29. The method of claim 26, wherein the first module and the second module comprise encoder models and the third module comprises a decoder model, optionally wherein the encoder models generate a high-dimensional representation for each DNA base in the target nucleic acid sequence and the decoder model generates predictions for each amino acid residue in the ZF protein sequence.

30. The method of claim 26, wherein the first module generates a first embedding and the second module generates a second embedding that are concatenated prior to being fed into the third module.

31. The method of claim 26, wherein the third module comprises at least one self-attention layer and at least one feed forward layer.

32. The method of claim 26, wherein the single helix binding data for training the first module and/or second module comprises data generated from bacterial one-hybrid (B1H) selection libraries.

33. The method of claim 26, wherein the single helix binding data for training the first module and/or second module comprises data on single ZF helices that bind polynucleotides comprising or consisting of a target 4-mer.

34. The method of claim 26, wherein the helix-pair binding data for training the third module comprises data generated from bacterial one-hybrid (B1H) selection libraries.

35. The method of claim 26, wherein the helix-pair binding data for training the third module comprises data on ZF-helix pairs that bind polynucleotides comprising or consisting of a target 7-mer.

36. The method of claim 26, wherein training one or more of the first module, the second module and the third module comprises providing a target nucleotide sequence and sequence of partially masked ZF residues and evaluating a cross-entropy loss based on output probabilities.


SEQUENCE LISTING (231 sequences)

SEQ ID NO: 1 - protein, 7 aa, synthetic construct
RSDNLRA

SEQ ID NO: 2 - protein, 7 aa, synthetic construct
QLATLSN

SEQ ID NO: 3 - protein, 7 aa, synthetic construct
DQSNLTR

SEQ ID NO: 4 - protein, 7 aa, synthetic construct
FQSGLIQ

SEQ ID NO: 5 - protein, 7 aa, synthetic construct
HKRNLTD

SEQ ID NO: 6 - protein, 7 aa, synthetic construct
DQSALLG

SEQ ID NO: 7 - protein, 7 aa, synthetic construct
TKQNLTH

SEQ ID NO: 8 - protein, 7 aa, synthetic construct
QLATLSY

SEQ ID NO: 9 - protein, 7 aa, synthetic construct
RNGNLTR

SEQ ID NO: 10 - protein, 7 aa, synthetic construct
YQPNLIN

SEQ ID NO: 11 - DNA, 21 bp, synthetic construct
  source 1..21: mol_type = other DNA
cgcccagctgggggcggggga

SEQ ID NO: 12 - protein, 7 aa, synthetic construct
QKSNLKR

SEQ ID NO: 13 - protein, 7 aa, synthetic construct
LKHHLTR

SEQ ID NO: 14 - protein, 7 aa, synthetic construct
DRSTLRQ

SEQ ID NO: 15 - protein, 7 aa, synthetic construct
DPSNLRR

SEQ ID NO: 16 - protein, 7 aa, synthetic construct
RKFNLLR

SEQ ID NO: 17 - protein, 7 aa, synthetic construct
DPSALNR

SEQ ID NO: 18 - protein, 7 aa, synthetic construct
RKDVLLG

SEQ ID NO: 19 - protein, 7 aa, synthetic construct
QSGTLYN

SEQ ID NO: 20 - protein, 7 aa, synthetic construct
RKAHLRD

SEQ ID NO: 21 - protein, 7 aa, synthetic construct
RKDRLRH

SEQ ID NO: 22 - protein, 7 aa, synthetic construct
WKCDLVR

SEQ ID NO: 23 - protein, 7 aa, synthetic construct
LKQTLQS

SEQ ID NO: 24 - protein, 7 aa, synthetic construct
RRGDLNR

SEQ ID NO: 25 - protein, 7 aa, synthetic construct
RKFTLRQ

SEQ ID NO: 26 - protein, 7 aa, synthetic construct
RRGTLRR

SEQ ID NO: 27 - protein, 6 aa, synthetic construct
RKWLNR

SEQ ID NO: 28 - protein, 7 aa, synthetic construct
TRRYLHS

SEQ ID NO: 29 - protein, 8 aa, synthetic construct
RDPSNLNR

SEQ ID NO: 30 - protein, 7 aa, synthetic construct
RRYGLRR

SEQ ID NO: 31 - protein, 7 aa, synthetic construct
HPSTLRN

SEQ ID NO: 32 - protein, 7 aa, synthetic construct
RKYSLLR

SEQ ID NO: 33 - protein, 7 aa, synthetic construct
FPYLLRN

SEQ ID NO: 34 - protein, 7 aa, synthetic construct
DRSTLRR

SEQ ID NO: 35 - protein, 8 aa, synthetic construct
DPSNLLRR

SEQ ID NO: 36 - protein, 6 aa, synthetic construct
KWTLKM

SEQ ID NO: 37 - protein, 7 aa, synthetic construct
RKFTLQC

SEQ ID NO: 38 - protein, 7 aa, synthetic construct
QSRYLRR

SEQ ID NO: 39 - protein, 7 aa, synthetic construct
QKSNLLR

SEQ ID NO: 40 - protein, 7 aa, synthetic construct
LKWNLKS

SEQ ID NO: 41 - protein, 7 aa, synthetic construct
RRDRLRH

SEQ ID NO: 42 - protein, 7 aa, synthetic construct
RKSTLQA

SEQ ID NO: 43 - protein, 7 aa, synthetic construct
RKYHLRQ

SEQ ID NO: 44 - protein, 7 aa, synthetic construct
RKSTLQS

SEQ ID NO: 45 - protein, 7 aa, synthetic construct
QLRYLSR

SEQ ID NO: 46 - protein, 7 aa, synthetic construct
TSSGLCH

SEQ ID NO: 47 - protein, 7 aa, synthetic construct
SKQNLQN

SEQ ID NO: 48 - protein, 7 aa, synthetic construct
RTSNLRR

SEQ ID NO: 49 - protein, 7 aa, synthetic construct
LKQTLTR

SEQ ID NO: 50 - protein, 7 aa, synthetic construct
RKWLTKM

SEQ ID NO: 51 - protein, 7 aa, synthetic construct
QSGNLRS

SEQ ID NO: 52 - protein, 7 aa, synthetic construct
HKRNLSA

SEQ ID NO: 53 - protein, 7 aa, synthetic construct
LKQTLSR

SEQ ID NO: 54 - protein, 7 aa, synthetic construct
RKWTLKM

SEQ ID NO: 55 - protein, 7 aa, synthetic construct
RKYHLQQ

SEQ ID NO: 56 - protein, 7 aa, synthetic construct
RKSTLQN

SEQ ID NO: 57 - protein, 7 aa, synthetic construct
RKSTLRS

SEQ ID NO: 58 - protein, 7 aa, synthetic construct
RRGDLLR

SEQ ID NO: 59 - protein, 7 aa, synthetic construct
FPYLLRR

SEQ ID NO: 60 - protein, 7 aa, synthetic construct
RRSTLRS

SEQ ID NO: 61 - protein, 7 aa, synthetic construct
YPYLLRA

SEQ ID NO: 62 - protein, 7 aa, synthetic construct
RRQTLRD

SEQ ID NO: 63 - protein, 7 aa, synthetic construct
RKSTLRD

SEQ ID NO: 64 - protein, 7 aa, synthetic construct
TSQSLSY

SEQ ID NO: 65 - protein, 7 aa, synthetic construct
YPYLLRR

SEQ ID NO: 66 - protein, 7 aa, synthetic construct
YPYLLRN

SEQ ID NO: 67 - protein, 7 aa, synthetic construct
RKQTLQD

SEQ ID NO: 68 - protein, 7 aa, synthetic construct
SRQSLNY

SEQ ID NO: 69 - protein, 7 aa, synthetic construct
QYSSLYK

SEQ ID NO: 70 - protein, 7 aa, synthetic construct
LKHHLLS

SEQ ID NO: 71 - protein, 7 aa, synthetic construct
QKAHLLR

SEQ ID NO: 72 - protein, 7 aa, synthetic construct
QKAHLSA

SEQ ID NO: 73 - protein, 7 aa, synthetic construct
RRYDLRM

SEQ ID NO: 74 - protein, 7 aa, synthetic construct
RRTTLRD

SEQ ID NO: 75 - protein, 7 aa, synthetic construct
QKVHLLR

SEQ ID NO: 76 - protein, 7 aa, synthetic construct
QKAHLTS

SEQ ID NO: 77 - protein, 7 aa, synthetic construct
RKYDLRM

SEQ ID NO: 78 - protein, 7 aa, synthetic construct
QKVHLRS

SEQ ID NO: 79 - protein, 7 aa, synthetic construct
QKSNLKT

SEQ ID NO: 80 - protein, 7 aa, synthetic construct
QKVDLNR

SEQ ID NO: 81 - protein, 7 aa, synthetic construct
YKFVLRS

SEQ ID NO: 82 - protein, 7 aa, synthetic construct
WRSSLVA

SEQ ID NO: 83 - protein, 7 aa, synthetic construct
YPYLLRH

SEQ ID NO: 84 - protein, 7 aa, synthetic construct
QKVHLLS

SEQ ID NO: 85 - protein, 7 aa, synthetic construct
RKGTLLR

SEQ ID NO: 86 - protein, 7 aa, synthetic construct
RKDTLLS

SEQ ID NO: 87 - protein, 7 aa, synthetic construct
QKAHLLA

SEQ ID NO: 88 - protein, 7 aa, synthetic construct
RKWNLLT

SEQ ID NO: 89 - protein, 7 aa, synthetic construct
VKRRLVN

SEQ ID NO: 90 - protein, 7 aa, synthetic construct
LKQTLQR

SEQ ID NO: 91 - protein, 7 aa, synthetic construct
QKVHLVT

SEQ ID NO: 92 - protein, 7 aa, synthetic construct
TSSHLCH

SEQ ID NO: 93 - protein, 7 aa, synthetic construct
RKWDLRQ

SEQ ID NO: 94 - protein, 7 aa, synthetic construct
RRSTLRD

SEQ ID NO: 95 - protein, 7 aa, synthetic construct
DPSNLNR

SEQ ID NO: 96 - protein, 7 aa, synthetic construct
RADRLRQ

SEQ ID NO: 97 - protein, 7 aa, synthetic construct
RTYNLLR

SEQ ID NO: 98 - protein, 7 aa, synthetic construct
DRTTLRR

SEQ ID NO: 99 - protein, 7 aa, synthetic construct
RRTHLRD

SEQ ID NO: 100 - protein, 7 aa, synthetic construct
RKDTLRN

SEQ ID NO: 101 - protein, 7 aa, synthetic construct
RKDTLRD

SEQ ID NO: 102 - protein, 7 aa, synthetic construct
RADRLRH

SEQ ID NO: 103 - protein, 7 aa, synthetic construct
RTTNLRR

SEQ ID NO: 104 - protein, 7 aa, synthetic construct
DPSNLLR

SEQ ID NO: 105 - protein, 7 aa, synthetic construct
RTYNLSR

SEQ ID NO: 106 - protein, 7 aa, synthetic construct
RRGDLRR

SEQ ID NO: 107 - protein, 7 aa, synthetic construct
RKGDLNR

SEQ ID NO: 108 - protein, 7 aa, synthetic construct
YPYLLRS

SEQ ID NO: 109 - protein, 7 aa, synthetic construct
QKVHLQS

SEQ ID NO: 110 - protein, 7 aa, synthetic construct
RKYHLSR

SEQ ID NO: 111 - protein, 7 aa, synthetic construct
RRYSLSA

SEQ ID NO: 112 - protein, 7 aa, synthetic construct
RKATLRD

SEQ ID NO: 113 - protein, 7 aa, synthetic construct
QKAHLTA

SEQ ID NO: 114 - protein, 7 aa, synthetic construct
RKWNLLR

SEQ ID NO: 115 - protein, 7 aa, synthetic construct
RRYSLRC

SEQ ID NO: 116 - protein, 7 aa, synthetic construct
RKFSLRN

SEQ ID NO: 117 - protein, 7 aa, synthetic construct
SSSNLLR

SEQ ID NO: 118 - protein, 7 aa, synthetic construct
LKHHLTS

SEQ ID NO: 119 - protein, 7 aa, synthetic construct
QKAHLSR

SEQ ID NO: 120 - protein, 7 aa, synthetic construct
RRDRLRS

SEQ ID NO: 121 - protein, 7 aa, synthetic construct
RKSNLLR

SEQ ID NO: 122 - protein, 7 aa, synthetic construct
QKAHLTR

SEQ ID NO: 123 - protein, 7 aa, synthetic construct
RRSTLRQ

SEQ ID NO: 124 - protein, 7 aa, synthetic construct
DRSTLRN

SEQ ID NO: 125 - protein, 7 aa, synthetic construct
RKDTLRS

SEQ ID NO: 126 - protein, 7 aa, synthetic construct
RKSNLNR

SEQ ID NO: 127 - protein, 7 aa, synthetic construct
ERSKLRR

SEQ ID NO: 128 - protein, 7 aa, synthetic construct
RRDRLRA

SEQ ID NO: 129 - protein, 7 aa, synthetic construct
RLYNLSR

SEQ ID NO: 130 - protein, 7 aa, synthetic construct
RRGDLHR

SEQ ID NO: 131 - protein, 7 aa, synthetic construct
RRGDLHR

SEQ ID NO: 132 - protein, 7 aa, Mus musculus
RKFTLLC

SEQ ID NO: 133 - protein, 7 aa, Mus musculus
RRDRLRN

SEQ ID NO: 134 - protein, 7 aa, Mus musculus
WKVDLKR

SEQ ID NO: 135 - protein, 7 aa, Mus musculus
RKFTLQS

SEQ ID NO: 136 - protein, 7 aa, Mus musculus
WKVDLSR

SEQ ID NO: 137 - protein, 7 aa, Mus musculus
RRDRLRR

SEQ ID NO: 138 - protein, 7 aa, Mus musculus
RRDRLLK

SEQ ID NO: 139 - protein, 7 aa, Mus musculus
DPSTLRR

SEQ ID NO: 140 - protein, 7 aa, Mus musculus
TSSNLSK

SEQ ID NO: 141 - protein, 7 aa, Mus musculus
HKHHLTG

SEQ ID NO: 142 - protein, 7 aa, Mus musculus
WKIDLLR

SEQ ID NO: 143 - protein, 7 aa, Mus musculus
RKWVLQC

SEQ ID NO: 144 - protein, 7 aa, Mus musculus
WKIDLVR

SEQ ID NO: 145 - protein, 7 aa, synthetic construct
RKAHLRE

SEQ ID NO: 146 - protein, 7 aa, synthetic construct
QKAHLKS

SEQ ID NO: 147 - protein, 7 aa, synthetic construct
RKWNLKM

SEQ ID NO: 148 - protein, 7 aa, synthetic construct
QKSHLTA

SEQ ID NO: 149 - protein, 7 aa, synthetic construct
RKWNLRM

SEQ ID NO: 150 - protein, 7 aa, synthetic construct
QSGTLHR

SEQ ID NO: 151 - protein, 7 aa, synthetic construct
RKWNLLM

SEQ ID NO: 152 - protein, 7 aa, synthetic construct
RKWTLLM

SEQ ID NO: 153 - protein, 7 aa, synthetic construct
RRAHLRD

SEQ ID NO: 154 - protein, 7 aa, synthetic construct
RKDRLLK

SEQ ID NO: 155 - protein, 7 aa, synthetic construct
QSSHLRR

SEQ ID NO: 156 - protein, 7 aa, synthetic construct
DRSNLRR

SEQ ID NO: 157 - protein, 7 aa, synthetic construct
TSSNLAH

SEQ ID NO: 158 - protein, 7 aa, synthetic construct
QSSHLTR

SEQ ID NO: 159 - protein, 7 aa, synthetic construct
RKWNLKQ

SEQ ID NO: 160 - protein, 7 aa, synthetic construct
RRDRLLS

SEQ ID NO: 161 - protein, 7 aa, synthetic construct
QKSNLRS

SEQ ID NO: 162 - protein, 7 aa, synthetic construct
RKAHLLS

SEQ ID NO: 163 - protein, 7 aa, synthetic construct
QKSHLKS

SEQ ID NO: 164 - protein, 7 aa, synthetic construct
QKVNLLS

SEQ ID NO: 165 - protein, 7 aa, synthetic construct
QKAHLTG

SEQ ID NO: 166 - protein, 7 aa, synthetic construct
HYKSLWR

SEQ ID NO: 167 - protein, 7 aa, synthetic construct
RKWTLKQ

SEQ ID NO: 168 - protein, 7 aa, synthetic construct
RRYHLSR

SEQ ID NO: 169 - protein, 7 aa, synthetic construct
QKVTLLR

SEQ ID NO: 170 - protein, 7 aa, synthetic construct
QRRYLTT

SEQ ID NO: 171 - protein, 7 aa, synthetic construct
QKSTLVR

SEQ ID NO: 172 - protein, 7 aa, synthetic construct
TKQYLSR

SEQ ID NO: 173 - protein, 7 aa, synthetic construct
QKAHLVR

SEQ ID NO: 174 - protein, 7 aa, synthetic construct
RKWTLSM

SEQ ID NO: 175 - protein, 6 aa, synthetic construct
NSTALQ

SEQ ID NO: 176 - protein, 6 aa, synthetic construct
ARNDSR

SEQ ID NO: 177 - protein, 6 aa, synthetic construct
RSDNRA

SEQ ID NO: 178 - protein, 6 aa, synthetic construct
QLATSN

SEQ ID NO: 179 - protein, 6 aa, synthetic construct
DQSNTR

SEQ ID NO: 180 - protein, 6 aa, synthetic construct
FQSGIQ

SEQ ID NO: 181 - protein, 6 aa, synthetic construct
HKRNTD

SEQ ID NO: 182 - protein, 6 aa, synthetic construct
DQSALG

SEQ ID NO: 183 - protein, 6 aa, synthetic construct
TKQNTH

SEQ ID NO: 184 - protein, 6 aa, synthetic construct
QLATSY

SEQ ID NO: 185 - protein, 6 aa, synthetic construct
RNGNTR

SEQ ID NO: 186 - protein, 6 aa, synthetic construct
YQPNIN

SEQ ID NO: 187 - protein, 6 aa, synthetic construct
RSDETR

SEQ ID NO: 188 - protein, 6 aa, synthetic construct
RKQTQT

SEQ ID NO: 189 - protein, 6 aa, synthetic construct
QKAHQT

SEQ ID NO: 190 - DNA, 21 bp, synthetic construct
  source 1..21: mol_type = other DNA
agtcgcccagctgggggcggg

SEQ ID NO: 191 - protein, 20 aa, synthetic construct
ACDEFGHIKLMNPQRSTVWY

SEQ ID NO: 192 - protein, 6 aa, synthetic construct
RTEDSR

SEQ ID NO: 193 - protein, 8 aa, synthetic construct
AEKNQRTV

SEQ ID NO: 194 - protein, 7 aa, synthetic construct
ADGNSTV

SEQ ID NO: 195 - protein, 8 aa, synthetic construct
ADHKNQRS

SEQ ID NO: 196 - protein, 4 aa, synthetic construct
KNRS

SEQ ID NO: 197 - protein, 7 aa, synthetic construct
ADHNQRT

SEQ ID NO: 198 - protein, 8 aa, synthetic construct
AEKNQRTV

SEQ ID NO: 199 - protein, 7 aa, synthetic construct
ADHNSTV

SEQ ID NO: 200 - protein, 6 aa, synthetic construct
ARNDSR

SEQ ID NO: 201 - protein, 6 aa, synthetic construct
NSTALQ

SEQ ID NO: 202 - protein, 6 aa, synthetic construct
RTNSQD

SEQ ID NO: 203 - protein, 6 aa, synthetic construct
QIGSQF

SEQ ID NO: 204 - protein, 6 aa, synthetic construct
NINPQY

SEQ ID NO: 205 - protein, 6 aa, synthetic construct
DTNRKH

SEQ ID NO: 206 - protein, 6 aa, synthetic construct
GLASQD

SEQ ID NO: 207 - protein, 6 aa, synthetic construct
HTNQKT

SEQ ID NO: 208 - protein, 6 aa, synthetic construct
YSTALQ

SEQ ID NO: 209 - protein, 6 aa, synthetic construct
RTNGNR

SEQ ID NO: 210 - protein, 6 aa, synthetic construct
RKQTQT

SEQ ID NO: 211 - protein, 6 aa, synthetic construct
QKAHQT

SEQ ID NO: 212 - DNA, 21 bp, synthetic construct
  source 1..21: mol_type = other DNA
aggagctgttcaccggggtgg

SEQ ID NO: 213 - DNA, 21 bp, synthetic construct
  source 1..21: mol_type = other DNA
tcctcgacaagtggccccacc

SEQ ID NO: 214 - DNA, 22 bp, synthetic construct
  source 1..22: mol_type = other DNA
gtctctatcactgatagggaga

SEQ ID NO: 215 - DNA, 21 bp, synthetic construct
  source 1..21: mol_type = other DNA
gtctctatcactgatagggag

SEQ ID NO: 216 - DNA, 21 bp, synthetic construct
  source 1..21: mol_type = other DNA
tctctatcactgatagggaga

SEQ ID NO: 217 - DNA, 22 bp, synthetic construct
  source 1..22: mol_type = other DNA
tctccctatcagtgatagagac

SEQ ID NO: 218 - DNA, 21 bp, synthetic construct
  source 1..21: mol_type = other DNA
tctccctatcagtgatagaga

SEQ ID NO: 219 - DNA, 21 bp, synthetic construct
  source 1..21: mol_type = other DNA
ctccctatcagtgatagagac

SEQ ID NO: 220 - protein, 42 aa, synthetic construct
QKVHLQSRKWTLSVRKGTLQDQYSSLYKRKGDLNKDPSSLRR

SEQ ID NO: 221 - protein, 42 aa, synthetic construct
RKYNLLRRRYSLSAQKAHLLSDPSNLRRQKRLLQNWKVDLRK

SEQ ID NO: 222 - protein, 42 aa, synthetic construct
RKFNLLRQSNTLRTLKHHLLNTSSGLCHEKRTLLNWKVDLRK

SEQ ID NO: 223 - protein, 42 aa, synthetic construct
QKTHLLTRRDYLTKRKFTLLRQSNDLRKLKQTLQDRRDRLRR

SEQ ID NO: 224 - protein, 341 aa, synthetic construct
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHSR

SEQ ID NO: 225 - protein, 374 aa, synthetic construct
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHSR

SEQ ID NO: 226 - protein, 393 aa, synthetic construct
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVPFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHI

SEQ ID NO: 227 - protein, 379 aa, synthetic construct
MDLKLKDCEFWYSLHGQVPGLLDWDMRNELFLPCTTDQCSLAEQILAKYRVGVMKPPEMPQKRRPSPDGDGPPCEPNLWMWVDPNILCPLGSQEAPKPSGKEDLTNISPFPQPPQKDEGSNCSEDKVVESLPSSSSEQSPLQKQGIHSPSDFELTEEEAEEPDDNSLQSPEMKCYQSQKLWQINNQEKSFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHIQECMSQPELLTSLFDL

SEQ ID NO: 228 - protein, 729 aa, synthetic construct
MDLPALLPAPTARGGQHGGGPGPLRRAPAPLGASPARRRLLLVRGPEDGGPGARPGEASGPSPPPAEDDSDGDSFLVLLEVPHGGAAAEAAGSQEAEPGSRVNLASRPEQGPSGPAAPPGPGVAPAGAVTISSQDLLVRLDRGVLALSAPPGPATAGAAAPRRAPQASGPSTPGFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHSRRQDLLPQLEAPSSLTPSSELSSPGQSELTNMDLAALFSDTPANASGSAGGSDEALNSGILTIDVTSVSSSLGGNLPANNSSLGPMEPLVLVAHSDIPPSLDSPLVLGTAATVLQQGSFSVDDVQTVSAGALGCLVALPMKNLSDDPLALTSNSNLAAHITTPTSSSTPRENASVPELLAPIKVEPDSPSRPGAVGQQEGSHGLPQSTLPSPAEQHGAQDTELSAGTGNFYLESGGSARTDYRAIQLAKEKKQRGAGSNAGASQSTQRKIKEGKMSPPHFHASQNSWLCGSLVVPSGGRPGPAPAAGVQCGAQGVQVQLVQDDPSGEGVLPSARGPATFLPFLTVDLPVYVLQEVLPSSGGPAGPEATQFPGSTINLQDLQ

SEQ ID NO: 229 - protein, 471 aa, synthetic construct
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLWYLSLEEVWKCRDQLDKYQENPERHLRQVAFTQKKVLTQERVSESGKYGGNCLLPAQLVLREYFHKRDSHTKSLKHDLVLNGHQDSCASNSNECGQTFCQNIHLIQFARTHTGDKSYKCPDNDNSLTHGSSLGISKGIHREKPFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGEQFLTCNQCGTALVNTSNLIGYQTNHIRENAY

SEQ ID NO: 230 - protein, 442 aa, synthetic construct
MAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSEGISLQGQVTQGNSVDSQLGQAEDQDGLSEMQEGHFRPGIDPQEKSPGKMSPECDGLGTADGVCSRIGQEQVSPGDRVRSHNSCESGKDPMIQEEENNFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGKNPISVTDVGRPFTSGQTSVTLRELLLLGKDFLNVTTEANILPEETSSSASDQPYQRETPQVSSL

SEQ ID NO: 231 - protein, 479 aa, synthetic construct
MAFEDVAVYFSQEEWGLLDTAQRALYRRVMLDNFALVASLGLSTSRPRVVIQLERGEEPWVPSGTDTTLSRTTYRRRNPGSWSLTEDRDVSGEWPRAFPDTPPGMTTSVFPAVGACHSVKSLQRQRGASPSRERKPTGVSVIYWERLLLGSGSGQASVSLRLTSPLRPPEGVRLREKTLTEHALLGRQPRTPERQKPCAQEVPGRTFGSAQDLEAAGGRGHHRMGAVWQEPHRLLGGQEPSTWDELGEALHAGEKSFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGEKTVRRSRASLHPQARSVAGASSEGAPAKETEPTPASGPAAVSQPAEV